(19) United States
(12) Patent Application Publication
(10) Pub. No.: US 2025/0318887 A1
(43) Pub. Date: Oct. 16, 2025

(54) EXTERNAL FORCE REGULATION FOR TELEOPERATION

(71) Applicant: AURIS HEALTH, INC., Santa Clara, CA (US)

(72) Inventors: Renbin ZHOU, Pleasanton, CA (US); Haoran YU, Sunnyvale, CA (US); Dennis MOSES, Santa Clara, CA (US); Nicholas CHEUNG, Redwood City, CA (US)

(21) Appl. No.: 19/251,673

(22) Filed: Jun. 26, 2025

Related U.S. Application Data
(63) Continuation of application No. PCT/IB2023/063391, filed on Dec. 29, 2023.
(60) Provisional application No. 63/436,466, filed on Dec. 30, 2022.

(51) Int. Cl. A61B 34/30; A61B 34/20; A61B 90/00

(52) U.S. Cl. CPC A61B 34/30; A61B 2034/2048; A61B 2090/031; A61B 2090/064

(57) ABSTRACT

Surgical robotic systems may regulate external forces for teleoperation. A surgical robot can include a surgical instrument configured to mount on a robotic arm. The surgical robot can estimate an external force applied to the surgical instrument during teleoperation while the surgical instrument or the robotic arm is in motion. The surgical robot can pause the motion of the surgical instrument or the robotic arm in response to detecting that the external force exceeds a first threshold. The surgical robot can reduce a velocity of the surgical instrument or the robotic arm in response to detecting that the external force exceeds a second threshold, which is lower than the first threshold.

### PRIORITY

This application is a continuation of International Patent Application No. PCT/IB2023/063391, filed Dec. 29, 2023, which claims priority to U.S. Provisional Patent Application No. 63/436,466, filed Dec. 30, 2022. The contents of all of the above-referenced patent applications are hereby incorporated by reference in their entirety for all purposes.

### TECHNICAL FIELD

The systems and methods disclosed herein are directed to devices and methods for indicating locations or orientations of surgical tools, and more particularly to surgical robotic systems for indicating locations or orientations of flexible surgical tools.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.).

Such robotic medical systems may include robotic arms configured to control the movement of surgical tool(s) during a given medical procedure. In order to achieve a desired pose of a surgical tool, a robotic arm may be placed into a particular pose during teleoperation. Some robotically enabled medical systems may include an arm support (e.g., a bar) that is connected to respective bases of the robotic arms and supports the robotic arms.

### SUMMARY

During robotic surgery, a robotic arm may, e.g., due to movement under teleoperation of the robotic arms, come into contact with adjacent objects such as another robotic arm, a patient, medical personnel, or accessories in the operating room, resulting in excessive contact force and/or torque on the patient or the medical personnel. The excessive contact force or torque may cause injury and discomfort to the patient or the medical personnel during surgery. In some circumstances, in response to such contact force and/or torque, one or more joints and/or links of the robotic arm may execute null space motion to maintain a pose (e.g., of a position and/or orientation of a cannula). In some circumstances, the operator may be required to move the patient or to reach for an input control before moving the robotic arm out of the way. However, these actions may pose additional risks of undesirable collisions and contact with the patient or other object in the operating room.

Accordingly, an improved robotic medical system is desirable. In particular, there is a need for a robotic medical system that detects interactions (e.g., external forces and/or torques) on a robotic arm (e.g., on linkages, joints, etc. of the robotic arm) and, depending on the characteristics (e.g., magnitude, direction, rate of change, etc.) of detected force and/or torque, take certain appropriate actions to regulate the external forces and/or torques.

As disclosed herein, a robotic control system causes movement of a robotic arm (or a portion thereof) in accordance with a command. The robotic control system monitors contact forces or torques exerted by an external object on the robotic arm during movement of the robotic arm. In accordance with a determination that the contact forces or torques meet a first set of conditions, the robotic control system reduces a velocity of the movement of the robotic arm that is being executed in accordance with the command. In some embodiments, in accordance with a determination that the one or more contact forces or torques meet a second set of conditions, the robotic control system stops the movement of the robotic arm that is being executed in accordance with the first command. Accordingly, the disclosed system and/or method advantageously improves patient and/or operator safety during surgery. It also ensures reduced interruption while the surgeon is driving one or more of the robotic arms during surgery.

In another aspect of the present disclosure, a robotic control system can provide feedback to a user in response to a determination that a robotic arm does not follow a commanded motion. In some embodiments, the feedback comprises haptic feedback to the user. In some embodiments, the feedback is in the form of a notification. In some embodiments, the feedback informs the user that a collision has occurred while still allowing the user to drive through a collision if the user deems it necessary or advisable (e.g., due to clinical reasons).

Accordingly, the disclosed systems and/or methods have several advantages over existing systems. For example, the disclosed system and/or methods provide feedback (e.g., haptic feedback or visual notifications) to a user in an informative and helpful manner, and can empower users to use the robotic medical system in a clinically useful manner (e.g., rather than having the robotic control system proactively prevent certain motions). The disclosed system also improves over predicate systems, which have low torque saturation and do not allow a surgeon to drive through an external collision, even if it may be clinically beneficial or necessary.

The systems, methods, and devices of this disclosure each have several innovative aspects, no single one of which is solely responsible for the desirable attributes disclosed herein.

In accordance with some embodiments of the present disclosure, a robotic control system includes one or more processors and memory. The memory stores instructions that when executed by the one or more processors, cause the one or more processors to perform operations for controlling a robotic arm. The operations include receiving a first command for moving at least a portion of the robotic arm. The operations include, in response to receiving the first command, causing movement of the at least a portion of the robotic arm in accordance with the first command and in accordance with a first set of conditions, including: monitoring one or more contact forces or torques exerted by an external object on the robotic arm during the movement of the robotic arm; and in accordance with a determination that the one or more contact forces or torques meet the first set of conditions, reducing a velocity of the movement of the robotic arm that is being executed in accordance with the first command.

In some embodiments, the one or more contact forces or torques include a first contact force. Reducing the velocity of the movement of the robotic arm includes determining a current velocity of the robotic arm, and reducing the velocity of the robotic arm from the current velocity to an updated velocity determined by a ratio based on the first contact force and an upper force limit.

In some embodiments, the operations further include repeating the steps of (i) determining the current velocity and (ii) reducing the velocity, until the magnitude of the first contact force is less than a lower force limit.

In some embodiments, the operations further include, in accordance with a determination that the one or more contact forces or torques meet a second set of conditions, stopping the movement of the robotic arm that is being executed in accordance with the first command.

In some embodiments, the operations further include, in response to receiving the first command, causing a motor of the robotic arm to generate a motor torque for initiating the movement of the at least a portion of the robotic arm.

In some embodiments, the operations further include calculating a first contact force based on the motor torque.

In some embodiments, the operations further include calculating a first contact torque based on the motor torque.

In some embodiments, the one or more contact forces or torques include a gravity torque due to gravitational forces on the robotic arm, and a friction torque due to frictional forces on the robotic arm.

In some embodiments, the one or more contact forces or torques include a dynamic torque, for balancing an inertia force and a Coriolis force of the robotic arm.

In some embodiments, the one or more contact forces or torques include a remote center of motion (RCM) torque, for constraining an instrument that is coupled to the robotic arm during teleoperation.

In some embodiments, the one or more contact forces or torques include a torque exerted by a patient on the robotic arm during teleoperation.

In some embodiments, the operations further include determining a teleoperation torque corresponding to a joint of the robotic arm according to a motor torque, the gravity torque, and the friction torque.

In some embodiments, the operations further include applying a filter to the teleoperation torque to obtain a filtered torque value; determining a force ratio according to the filtered torque value; and using the determined force ratio as an input for an inverse kinematic solver.

In some embodiments, the operations further include, in accordance with a determination that the filtered torque value is less than a lower joint torque limit, designating the force ratio as zero. The operations further include, in accordance with a determination that the filtered torque value is between the lower joint torque limit and an upper joint torque limit, determining the force ratio according to a ratio between (i) the square of a difference between the filtered torque value and the lower joint torque limit and (ii) the square of a difference between the upper joint torque limit and the lower joint torque limit. The operations further include, in accordance with a determination that the filtered torque value is greater than or equal to the upper joint torque limit, designating the force ratio as one.

In some embodiments, the operations further include determining the lower joint torque limit and the upper joint torque limit corresponding to the joint of the robotic arm based on the motor torque.

In some embodiments, the first command includes a first commanded position for the robotic arm. The operations further include, in accordance with a determination that a difference between the first commanded position and an actual position of the robotic arm meet first criteria, the first criteria including a first threshold amount of difference, generating and outputting a notification regarding the difference.

In some embodiments, generating and outputting the notification includes causing the notification to be output as haptic feedback to a user.

In some embodiments, generating and outputting the notification includes causing the notification to be displayed on a display device.

In some embodiments, the first command includes a first commanded position for the robotic arm. The operations further include, after reducing the velocity of the movement of the robotic arm, causing second movement of the at least a portion of the robotic arm to the first commanded position; and generating and outputting a notification regarding the second movement.

In some embodiments, generating and outputting the notification regarding the second movement includes causing the notification to be output as haptic feedback to a user.

In accordance with some embodiments of the present disclosure, a method is performed at a robotic control system having one or more processors and memory. The method includes receiving a first command for moving at least a portion of a robotic arm. The method includes, in response to receiving the first command, causing movement of the at least a portion of the robotic arm in accordance with the first command and in accordance with a first set of conditions, including: monitoring one or more contact forces or torques exerted by an external object on the robotic arm during the movement of the robotic arm; and in accordance with a determination that the one or more contact forces or torques meet the first set of conditions, reducing a velocity of the movement of the robotic arm that is being executed in accordance with the first command.

In some embodiments, the one or more contact forces or torques include a first contact force. Reducing the velocity of the movement of the robotic arm includes determining a current velocity of the robotic arm; and reducing the velocity of the robotic arm from the current velocity to an updated velocity determined by a ratio based on the first contact force and an upper force limit.

In some embodiments, the method further includes repeating the steps of (i) determining the current velocity and (ii) reducing the velocity, until the magnitude of the first contact force is less than a lower force limit.

In some embodiments, the method further includes, in accordance with a determination that the one or more contact forces or torques meet a second set of conditions, stopping the movement of the robotic arm that is being executed in accordance with the first command.

In some embodiments, the robotic arm includes a motor. The method further includes in response to receiving the first command, causing a motor of the robotic arm to generate a motor torque for initiating the movement of the at least a portion of the robotic arm.

In some embodiments, the method further includes calculating a first contact force based on the motor torque.

In some embodiments, the method further includes calculating a first contact torque based on the motor torque.

In some embodiments, the one or more contact forces or torques include a gravity torque due to gravitational forces on the robotic arm and a friction torque due to frictional forces on the robotic arm.

In some embodiments, the one or more contact forces or torques include a dynamic torque, for balancing an inertia force and a Coriolis force of the robotic arm.

In some embodiments, the one or more contact forces or torques include a remote center of motion (RCM) torque, for constraining an instrument that is coupled to the robotic arm during teleoperation.

In some embodiments, the one or more contact forces or torques include a torque exerted by a patient on the robotic arm during teleoperation.

In some embodiments, the method further includes determining a teleoperation torque corresponding to a joint of the robotic arm according to a motor torque, the gravity torque, and the friction torque.

In some embodiments, the method further includes applying a filter to the teleoperation torque to obtain a filtered torque value. The method further includes determining a force ratio according to the filtered torque value. The method further includes using the determined force ratio as an input for an inverse kinematic solver.

In some embodiments, the method further includes, in accordance with a determination that the filtered torque value is less than a lower joint torque limit, designating the force ratio as zero. The method further includes, in accordance with a determination that the filtered torque value is between the lower joint torque limit and an upper joint torque limit, determining the force ratio according to a ratio between (i) the square of a difference between the filtered torque value and the lower joint torque limit and (ii) the square of a difference between the upper joint torque limit and the lower joint torque limit. The method further includes, in accordance with a determination that the filtered torque value is greater than or equal to the upper joint torque limit, designating the force ratio as one.

In some embodiments, the method further includes determining the lower joint torque limit and the upper joint torque limit corresponding to the joint of the robotic arm based on the motor torque.

In some embodiments, the first command includes a first commanded position for the robotic arm. The method further includes, in accordance with a determination that a difference between the first commanded position and an actual position of the robotic arm meet first criteria, the first criteria including a first threshold amount of difference, generating and outputting a notification regarding the difference.

In some embodiments, generating and outputting the notification includes causing the notification to be output as haptic feedback to a user.

In some embodiments, generating and outputting the notification includes causing the notification to be displayed on a display device.

In some embodiments, the first command includes a first commanded position for the robotic arm. The method further includes, after reducing the velocity of the movement of the robotic arm, causing second movement of the at least a portion of the robotic arm to the first commanded position. The method further includes generating and outputting a notification regarding the second movement.

In some embodiments, generating and outputting the notification regarding the second movement includes causing the notification to be output as haptic feedback to a user.

In accordance with some embodiments of the present disclosure, a surgical robot includes a surgical instrument configured to mount on a robotic arm. The surgical robot includes a processor. The processor is configured to estimate an external force applied to the surgical instrument during teleoperation while the surgical instrument or the robotic arm is in motion. The processor is configured to, in response to detecting the external force exceeding a first threshold, pause the motion of the surgical instrument or the robotic arm. The processor is configured to, in response to detecting the external force exceeding a second threshold, which is lower than the first threshold, reduce a velocity of the surgical instrument or the robotic arm.

In some embodiments, the external force is applied through contact with at least one of another surgical instrument, another robotic arm, a surgical table, a medical device, a patient, or a medical personnel.

In some embodiments, the motion of the surgical instrument or the robotic arm is driven by at least one of a plurality of actuators. Estimating the external force includes calculating a motor torque at each of the plurality of actuators based on a motor current and a gear ratio.

In some embodiments, the motor torque includes a regular torque for teleoperation and an external torque to balance the external force.

In some embodiments, the regular torque for teleoperation comprises one or more of: a gravity compensation torque, a dynamic torque to balance inertia and Coriolis effect, a friction torque, a remote center of motion (RCM) torque, and a tissue load torque.

In some embodiments, the external torque to balance the external force is estimated based on one or more of: the calculated motor torque, a pose, a velocity and an acceleration of the robotic arm and surgical instrument, a maximum expected RCM torque, and a maximum expected tissue load torque.

In some embodiments, the external force is estimated at a tooltip of the surgical instrument.

In some embodiments, the processor is further configured to gradually reduce the motion of the surgical instrument or the robotic arm following a smooth curve (e.g., based on a smoothing function).

In some embodiments, the first threshold and the second threshold are predetermined.

In some embodiments, at least one of the first threshold and the second threshold is determined in real time based on one or more of a pose, a velocity and an acceleration of the surgical instrument or the robotic arm.

In some embodiments, the processor is further configured to output a notification on detecting the external force exceeding the first threshold or the second threshold, wherein the notification includes haptic feedback and/or audio-visual warnings.

In accordance with some embodiments of the present disclosure, a computer-implemented method comprises estimating an external force applied at a position on a surgical manipulator while the surgical manipulator is in motion during a teleoperated robotic surgery. The method includes determining whether the external force is excessive by comparing the external force to a slowdown force threshold. The method includes, in response to determining the external force is excessive, slowing down the motion of the surgical manipulator and generating a notification about the excessive external force.

In some embodiments, the external force is caused by a contact with other objects around the surgical manipulator.

In some embodiments, the external force is estimated based on a difference between an actual force and a maximum expected force for teleoperated robotic surgery at the position on the surgical manipulator.

In some embodiments, the position on the surgical manipulator for estimating the external force includes any positions at a tooltip, a tool shaft, a tool stage, a tool driver, and a robotic arm.

In some embodiments, the slowdown threshold is predetermined or determined in real time based on a pose and/or motion status of the surgical manipulator.

In some embodiments, the method further includes determining whether the external force is excessive over a stop force threshold, which is higher than slowdown force threshold. The method includes, in response to determining the external force is excessive over the stop threshold, stopping the motion of the surgical manipulator and generating a notification about the excessive external force.

In some embodiments, the method further includes repeating the steps of (i) estimating the external force at the position on the surgical manipulator, and (ii) slowing down the motion of the surgical manipulator, until the external force at the position falls below the slowdown threshold.

In some embodiments, the notification about the excessive external force includes haptic feedback and/or audio-visual warnings.

In accordance with some embodiments of the present disclosure, a non-transitory computer readable storage medium stores computer-executable instructions, when executed by one or more processors of a robotic control system, cause the one or more processors to perform operations including: estimating an external force applied at a position on a robotic manipulator while the robotic manipulator is in motion during a teleoperated surgery, the external force caused by a contact with other objects around the robotic manipulator; determining whether the external force is in excess of a safety threshold; and in response to determining the external force is in excess of the safety threshold, stopping the motion of the robotic manipulator; and generating a notification including haptic feedback and/or audio-visual warnings about the external force in excess of the safety threshold.

In accordance with some embodiments of the present disclosure, a robotic control system includes one or more processors and memory. The memory stores instructions that, when executed by the one or more processors, cause the one or more processors to perform any of the methods disclosed herein.

In accordance with some embodiments of the present disclosure, a non-transitory computer readable storage medium stores computer-executable instructions. The computer-executable instructions, when executed by one or more processors of a robotic control system, cause the one or more processors to perform any of the methods disclosed herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an exemplary robotic system according to some embodiments.

FIG. 2 illustrates another view of an exemplary robotic system according to some embodiments.

FIGS. 3A and 3B illustrate different views of an exemplary robotic surgical system according to some embodiments.

FIG. 4 illustrates an exemplary view of a robotic surgical system with the robotic arms in a stowed position, in accordance with some embodiments.

FIG. 5 illustrates components of a robotic medical system in accordance with some embodiments.

FIGS. 6A to 6C illustrate different views of an exemplary robotic arm according to some embodiments.

FIGS. 7A and 7B illustrate different views of an exemplary robotic surgical system according to some embodiments.

FIG. 8 illustrates a part of a robotic arm 350 and a surgical tool 360 according to some embodiments.

FIG. 9 illustrates a perspective view of a robotic medical system in accordance with some embodiments.

FIGS. 10A to 10D illustrate a flowchart diagram for a method performed by one or more processors of a robotic system, in accordance with some embodiments.

FIGS. 11A and 11B illustrate a flowchart diagram for a method performed by one or more processors of a robotic system, in accordance with some embodiments.

FIGS. 12A and 12B illustrate a flowchart diagram for a method performed by one or more processors of a robotic system, in accordance with some embodiments.

FIG. 13 illustrates a flowchart diagram for a method performed by one or more processors of a robotic system, in accordance with some embodiments.

FIG. 14 is a schematic diagram illustrating electronic components of a robotic medical system in accordance with some embodiments.

### DETAILED DESCRIPTION

### 1. Overview

Aspects of the present disclosure may be integrated into a robotically enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved case of use such that one or more of the instruments of the system can be controlled by a single user.

Various embodiments will be described below in conjunction with the drawings for purposes of illustration. It should be appreciated that many other embodiments of the disclosed concepts are possible, and various advantages can be achieved with the disclosed embodiments. Headings are included herein for reference and to aid in locating various sections. These headings are not intended to limit the scope of the concepts described with respect thereto. Such concepts may have applicability throughout the entire specification.

### 2. External Force Regulation for Teleoperation

This application discloses robotic control systems that regulate external forces for teleoperation, e.g., in minimally invasive surgeries. A robotic control system receives a first command for moving at least a portion of the robotic arm. In response to receiving the first command, the system causes movement of the at least a portion of the robotic arm in accordance with the first command and in accordance with a first set of conditions. The system can monitor one or more contact forces or torques exerted by an external object on the robotic arm during the movement of the robotic arm. In some embodiments, in accordance with a determination that the one or more contact forces or torques meet the first set of conditions, the system can reduce a velocity of the movement of the robotic arm that is being executed in accordance with the first command.

In some embodiments, the system can stop the movement of the robotic arm that is being executed in accordance with the first command in accordance with a determination that the one or more contact forces or torques meet a second set of conditions.

In some embodiments, the system can, in accordance with a determination that there is a mismatch between a commanded position and an actual position of a robotic arm (e.g., a joint or a link of the robotic arm), generate and output (or cause output of) feedback to a user regarding the difference.

In some embodiments, the feedback includes haptic feedback.

In some embodiments, the feedback includes visual feedback.

### A. Robotic System.

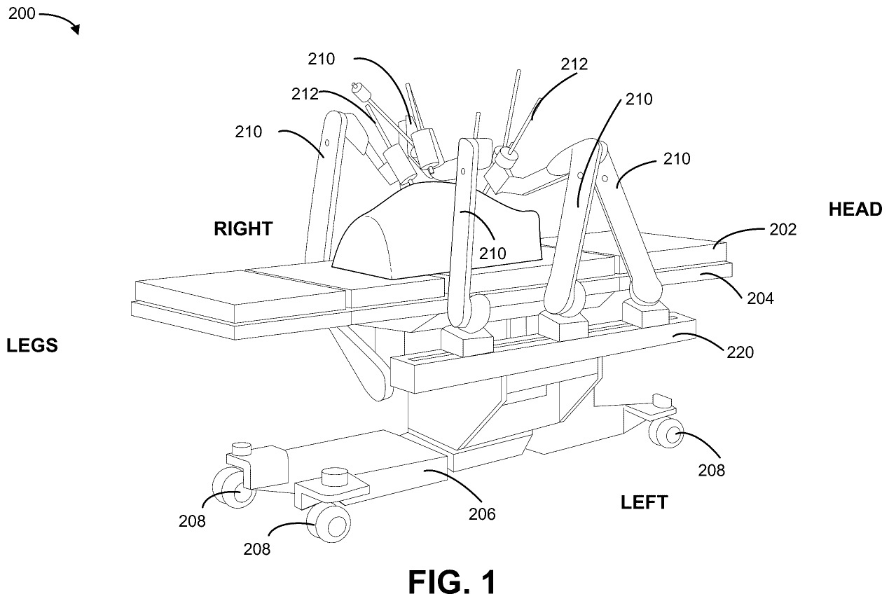

FIG. 1 illustrates an exemplary robotic medical system 200 according to some embodiments. In some embodiments, the robotic medical system 200 is a robotic surgery system. In the example of FIG. 1, the robotic medical system 200 comprises a patient support platform 202 (e.g., a patient platform, a table, a bed, etc.). The two ends along the length of the patient support platform 202 are respectively referred to as “head” and “leg”. The two sides of the patient support platform 202 are respectively referred to as “left” and “right.” The patient support platform 202 includes a support 204 (e.g., a rigid frame) for the patient support platform 202.

The robotic medical system 200 also includes a base 206 for supporting the robotic medical system 200. The base 206 includes wheels 208 that allow the robotic medical system 200 to be easily movable or repositionable in a physical environment. In some embodiments, the wheels 208 are omitted from the robotic medical system 200 or are retractable, and the base 206 can rest directly on the ground or floor. In some embodiments, the wheels 208 are replaced with feet.

The robotic medical system 200 includes one or more robotic arms 210. In some embodiments, the robotic arms 210 can be configured to perform robotic medical procedures. Although FIG. 1 shows five robotic arms 210, it should be appreciated that the robotic medical system 200 may include any number of robotic arms, including less than five or six or more.

The robotic medical system 200 also includes one or more bars 220 (e.g., adjustable arm support or an adjustable bar) that support the robotic arms 210. Each of the robotic arms 210 is supported on, and movably coupled to, a bar 220, by a respective base joint of the robotic arm. In some embodiments, bar 220 can provide several degrees of freedom, including lift, lateral translation, tilt, etc. In some embodiments, each of the robotic arms 210 and/or the adjustable arm supports 220 is also referred to as a respective kinematic chain.

FIG. 1 shows three robotic arms 210 supported by the bar 220 that is in the field of view of the figure. The two remaining robotic arms are supported by another bar that is located across the other length of the patient support platform 202.

In some embodiments, the adjustable arm supports 220 can be configured to provide a base position for one or more of the robotic arms 210 for a robotic medical procedure. A robotic arm 210 can be positioned relative to the patient support platform 202 by translating the robotic arm 210 along a length of its underlying bar 220 and/or by adjusting a position and/or orientation of the robotic arm 210 via one or more joints and/or links. In some embodiments, the bar pose can be changed via manual manipulation, teleoperation, and/or power assisted motion.

In some embodiments, the adjustable arm support 220 can be translated along a length of the patient support platform 202. In some embodiments, translation of the bar 220 along a length of the patient support platform 202 causes one or more of the robotic arms 210 supported by the bar 220 to be simultaneously translated with the bar or relative to the bar. In some embodiments, the bar 220 can be translated while keeping one or more of the robotic arms stationary with respect to the base 206 of the robotic medical system 200.

In the example of FIG. 1, the adjustable arm support 220 is located along a length of the patient support platform 202. In some embodiments, the adjustable arm support 220 may extend across a partial or full length of the patient support platform 202, and/or across a partial or full width of the patient support platform 202.

During a robotic medical procedure, one or more of the robotic arms 210 can also be configured to hold instruments 212 (e.g., robotically controlled medical instruments or tools, such as an endoscope and/or any other instruments (e.g., sensors, illumination instrument, cutting instrument, etc.) that may be used during surgery), and/or be coupled to one or more accessories, including one or more cannulas, in accordance with some embodiments.

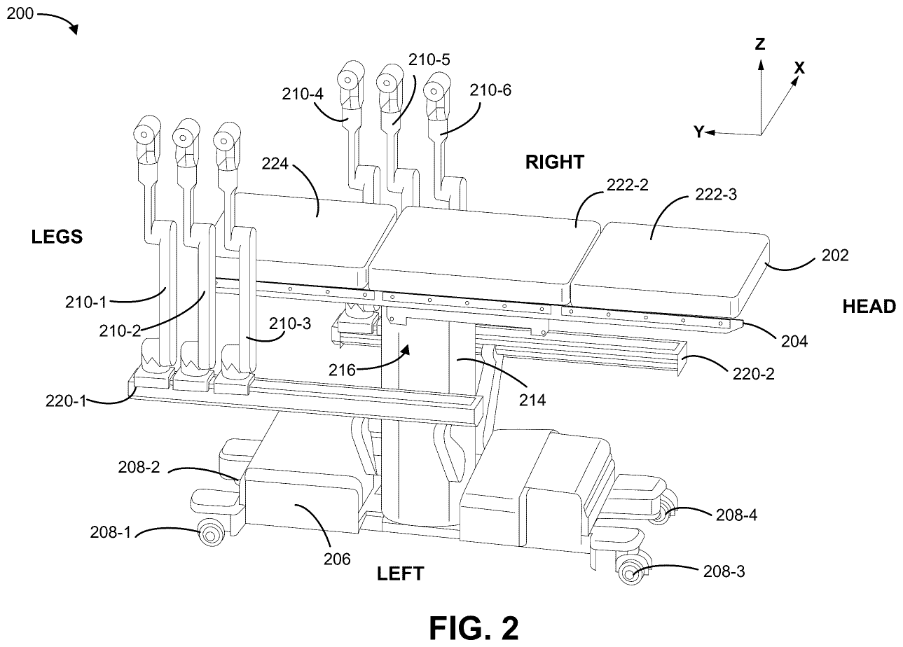

FIG. 2 illustrates another view of the exemplary robotic medical system 200 in FIG. 1 according to some embodiments. In this example, the robotic medical system 200 includes six robotic arms 210-1, 210-2, 210-3, 210-4, 210-5, and 210-6. The patient platform 202 is supported by a column 214 that extends between the base 206 and the patient platform 202. In some embodiments, the patient platform 202 comprises a tilt mechanism 216. The tilt mechanism 216 can be positioned between the column 214 and the patient platform 202 to allow the patient platform 202 to pivot, rotate, or tilt relative to the column 214. The tilt mechanism 216 can be configured to allow for lateral and/or longitudinal tilt of the patient platform 202. In some embodiments, the tilt mechanism 216 allows for simultaneous lateral and longitudinal tilt of the patient platform 202.

FIG. 2 shows the patient platform 202 in an untilted state or position. In some embodiments, the untilted state or position is a default position of the patient platform 202. In some embodiments, the default position of the patient platform 202 is a substantially horizontal position as shown in FIG. 2. As illustrated, in the untilted state, the patient platform 202 can be positioned horizontally or parallel to a surface that supports the robotic medical system 200 (e.g., the ground or floor). In some embodiments, the term “untilted” refers to a state in which the angle between the default position and the current position is less than a threshold angle (e.g., less than 5 degrees, or less than an angle that would cause the patient to shift on the patient platform, etc.). In some embodiments, the term “untilted” refers to a state in which the patient platform is substantially perpendicular to the direction of gravity, irrespective of the angle formed by the surface that supports the robotic medical system relative to gravity.

With continued reference to FIG. 2, in the illustrated example of the robotic medical system 200, the patient platform 202 includes a support 204. In some embodiments, the support 204 includes a rigid support structure or frame, and can support one or more surfaces, pads, or cushions 222. An upper surface of the patient platform 202 can include a support surface 224. During a medical procedure, a patient can be placed on the support surface 224.

FIG. 2 shows the robotic arms 210 and the adjustable arm supports 220 in an exemplary deployed configuration in which the robotic arms 210 reach above the patient platform 202. In some embodiments, due to the configuration of the robotic medical system 200, which enables stowage of different components beneath the patient platform 202, the robotic arms 210 and the arm supports 220 can occupy a space underneath the patient platform 202. Thus, in some embodiments, the tilt mechanism 216 has a low-profile and/or low volume in order to increase the space available for storage below.

FIG. 2 also illustrates an example, x, y, and z coordinate system that may be used to describe certain features of the embodiments disclosed herein. It will be appreciated that this coordinate system is provided for purposes of example and explanation only and that other coordinate systems may be used. In the illustrated example, the x-direction or x-axis extends in a lateral direction across the patient platform 202 when the patient platform 202 is in an untilted state. In some configurations, the x-direction extends across the patient platform 202 from one lateral side (e.g., the right side) to the other lateral side (e.g., the left side) when the patient platform 202 is in an untilted state. The y-direction or y-axis extends in a longitudinal direction along the patient platform 202 when the patient platform 202 is in an untilted state. That is, the y-direction extends along the patient platform 202 from one longitudinal end (e.g., the head end) to the other longitudinal end (e.g., the legs end) when the patient platform 202 is in an untilted state. In an untilted state, the patient platform 202 can lie in or be parallel to the x-y plane, which can be parallel to the floor or ground. In the illustrated example, the z-direction or z-axis extends along the column 214 in a vertical direction. In some embodiments, the tilt mechanism 216 is configured to laterally tilt the patient platform 202 by rotating the patient platform 202 about a lateral tilt axis that is parallel to the y-axis. The tilt mechanism 216 can further be configured to longitudinally tilt the patient platform 202 by rotating the patient platform 202 about a longitudinal tilt axis that is parallel to the x-axis.

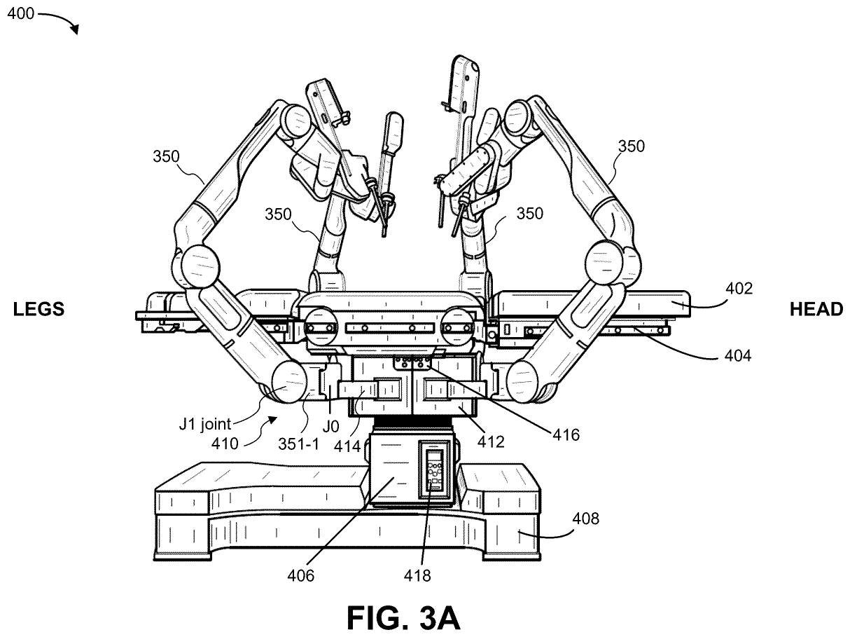

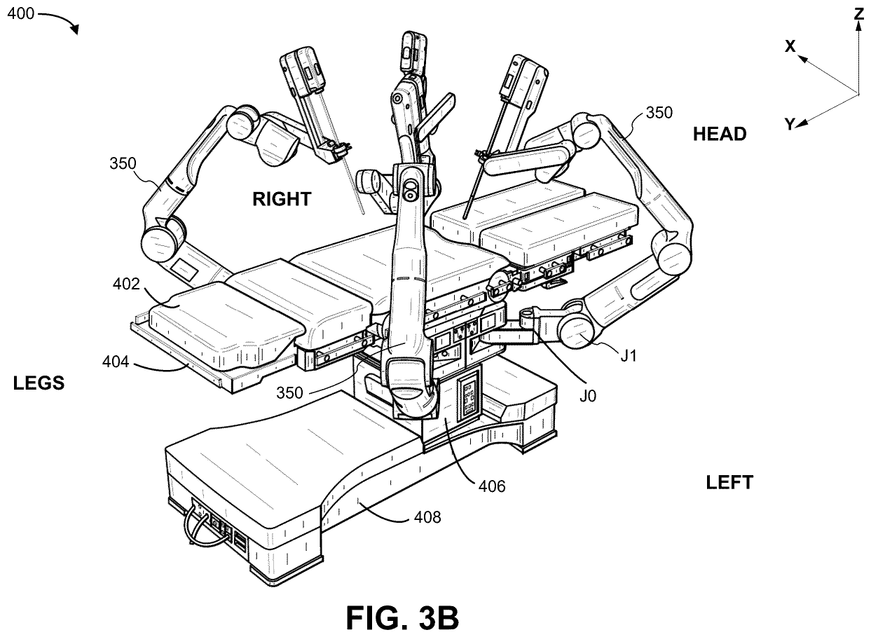

FIGS. 3A and 3B illustrate different views of an exemplary robotic surgical system 400 (e.g., robotic medical system) according to some embodiments. The robotic surgical system 400 includes a surgical table having a table top 402 on which a patient can be disposed. The table top 402 is supported by a table adapter 404. The robotic surgical system 400 includes a support 406 (e.g., a support mechanism, a table column or a pedestal) and a base 408. The support 406 may be mounted to the base 408, which can be fixed to the floor of an operating room, or can be movable relative to the floor, e.g., by use of wheels on the base 408. In some embodiments, the various sections of the table top 402 can move relative to each other (e.g., can be tilted or angled relative to each other) and/or the table top 402 can be moved (e.g., tilted, angled) relative to the support 406 and/or the base 408 of the surgical table.

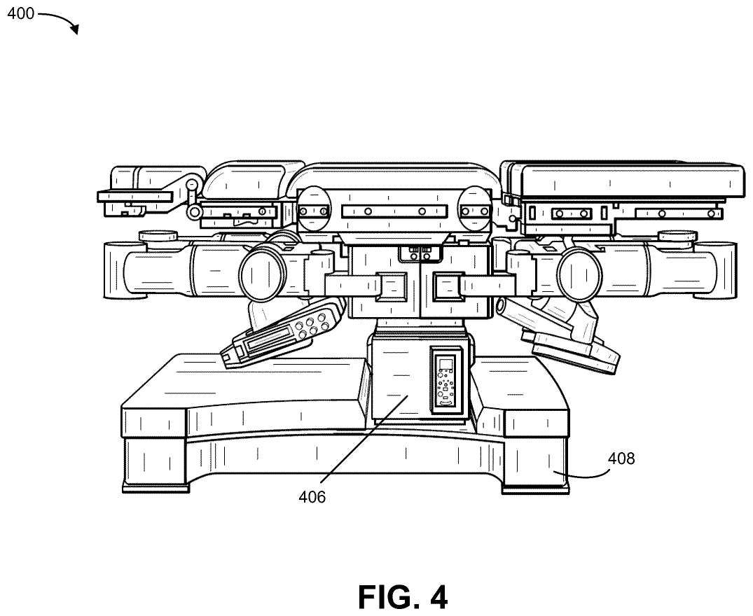

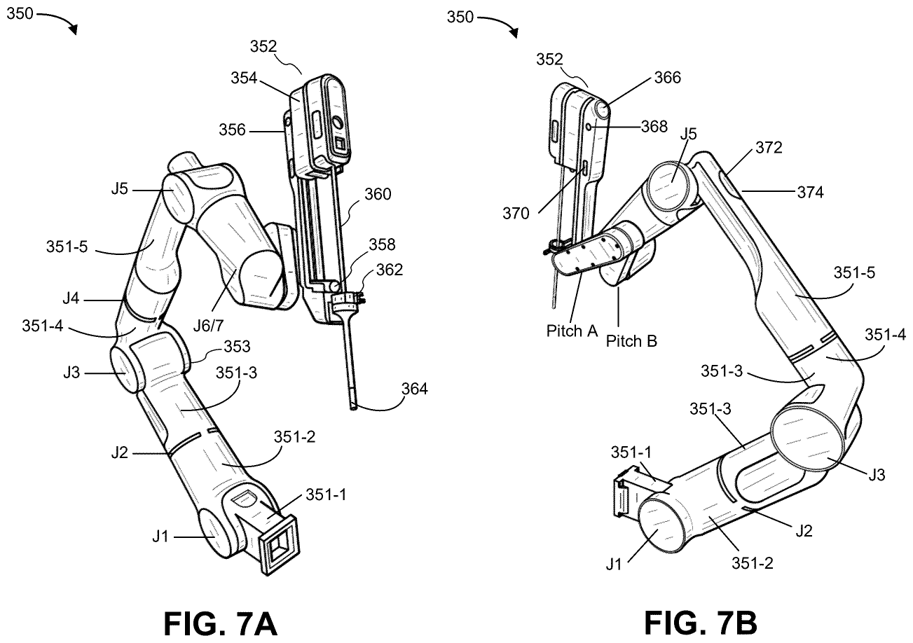

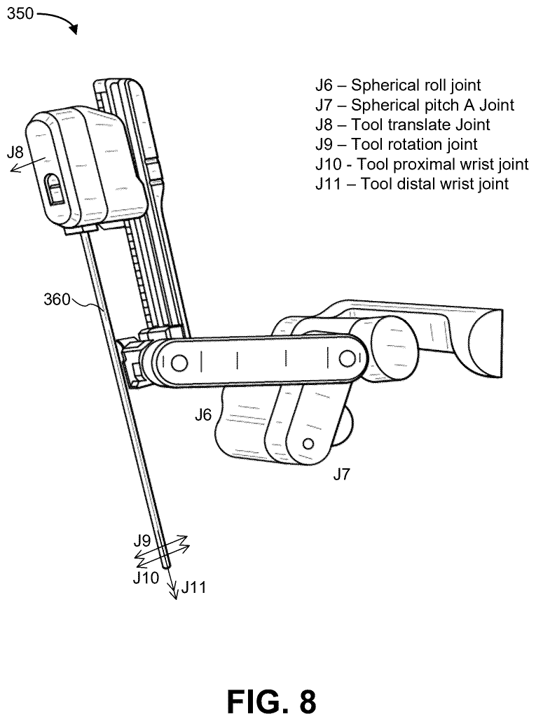

In some embodiments, the robotic surgical system 400 includes robotic arms 350 coupled, or coupleable to, the surgical table. The robotic arms 350 can be moved between multiple different positions relative to the surgical table, such as, for example, an operating position, a parked position, or a stowed position (e.g., as illustrated in FIG. 4). A robotic arm 350 can support a medical instrument or tool, such as a surgical instrument, tool driver, and/or imaging device. Further details of the robotic arm 350 are described in FIGS. 7A, 7B, and 8.

In some embodiments, the robotic surgical system 400 includes one or more input devices (e.g., buttons, switches, touch-sensitive surfaces, etc.), such as a pivot and stow keypad 416 and a table keypad 418.

In some embodiments, a proximal portion of the robotic arm 350 can be implemented as an adapter 410, which may be fixedly coupled to the surgical table. The adapter 410 can include an interface mechanism 412 (e.g., table interface structure), a first link member (e.g., link 414) pivotally coupled to the interface mechanism 412 at a first joint (e.g., J0 joint), and coupled to a second link member (e.g., link 351-1) at a second joint (e.g., J1 joint). In some embodiments, the second link member can be pivotally coupled to the first link member at the second joint. In some embodiments, the second link member is slidably coupled to the first link member at the second joint. The second link member is also configured to be coupled to a robotic arm at a coupling that includes a coupling portion of the second link member and a coupling portion at a proximal or mounting end portion of the robotic arm. The robotic arm also includes a target joint at the mounting end portion of the robotic arm. In some embodiments, the target joint is included with the coupling portion at the mounting end portion of the robotic arm.

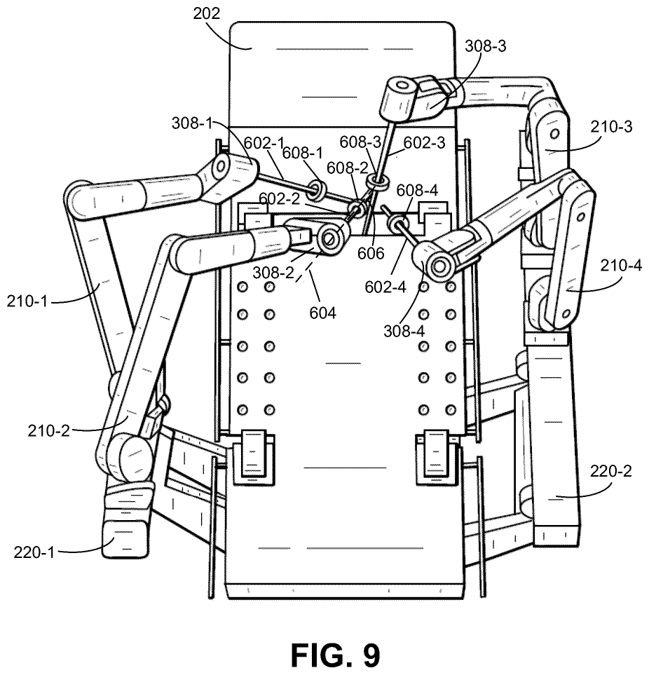

In some embodiments, the distinction between an adapter 410 and robotic arm 350 can be disregarded, and the connection between the surgical table and a distal end of the robotic arm 350 can be conceptualized and implemented as a series of links and joints that provide the desired degrees of freedom for movement of the medical instrument. Details of the links and joints of the robotic arm 350 are described with respect to FIGS. 8A, 8B, and 9.

FIG. 4 illustrates an exemplary view of a robotic surgical system 400 with the robotic arms 350 in a stowed position, in accordance with some embodiments.

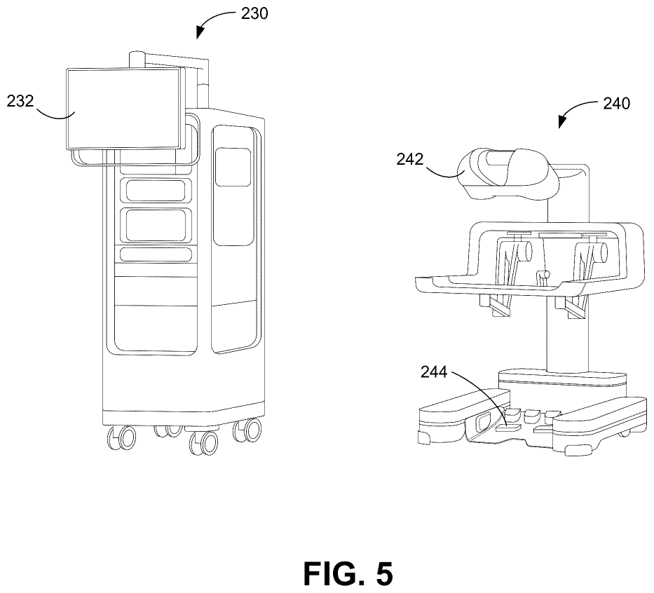

In some embodiments, the robotic medical system 200 or the robotic surgical system 400 includes a tower 230 (e.g., tower viewer) or a physician console 240 (or both), as illustrated in FIG. 5. The tower 230 may provide support for controls, electronics, fluidics, optics, sensors, and/or power for the patient support platform 202 and the physician console 240. In some embodiments, the tower 230 includes a display device 232. The display device 232 can include a user interface for displaying a surgical view obtained by one or more cameras 606 of the robotic medical system and/or one or more notifications to an operator of the robotic medical system 200. In some embodiments, the physician console 240 can include a display device 242 having a user interface used by the physician operator for operating the patient support platform 202. For example, the display device 242 may include a user interface for displaying a surgical view obtained by one or more cameras 606 of the robotic medical system and/or one or more notifications to an operator of the robotic medical system 200. The physician console 240 can provide both robotic controls and pre-operative and real-time information of a medical procedure to a physician operator. In some embodiments, the physician console 240 includes one or more input devices (e.g., buttons, switches, touch-sensitive surfaces, gimbals, etc.), such as a foot pedal 244. In some embodiments, the physician console 240 includes one or more haptic interface devices (HID) that provide force and tactile feedback to a user as the user interacts with the physician console 240.

### B. Robotic Arm.

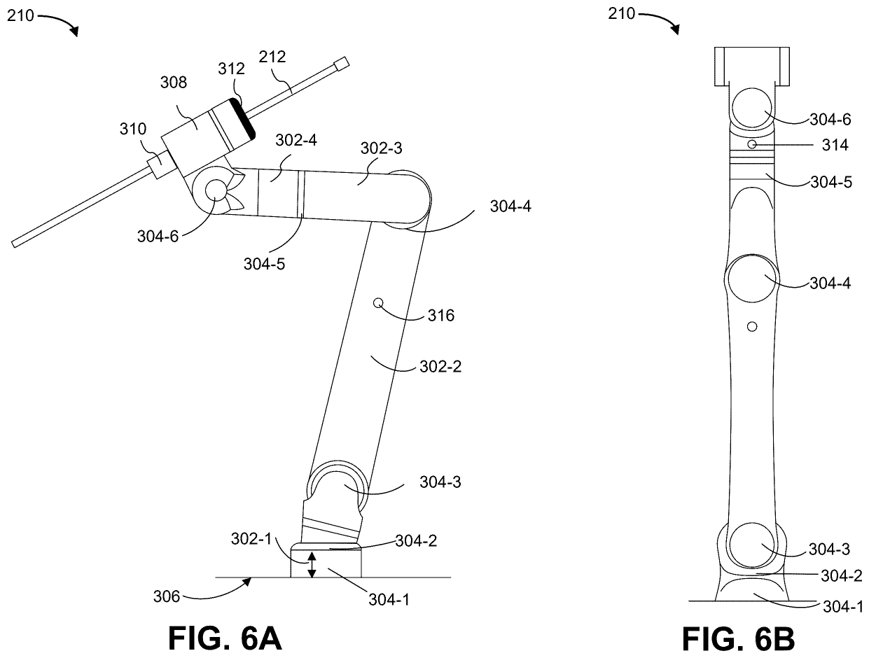

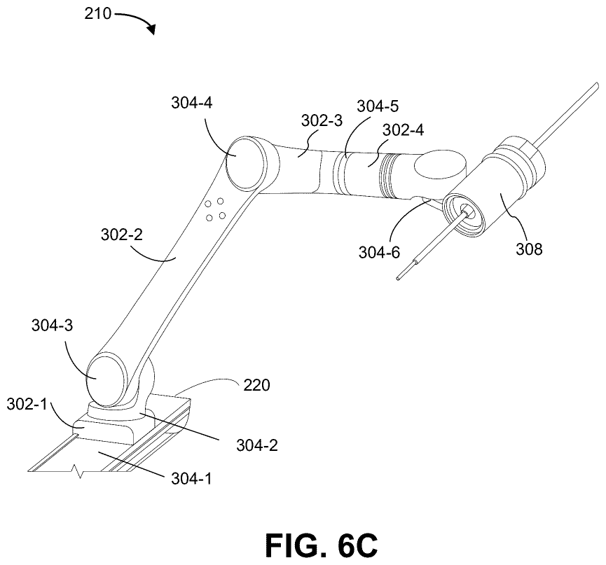

FIGS. 6A, 6B, and 6C illustrate different views of an exemplary robotic arm 210 according to some embodiments.

FIG. 6A illustrates that the robotic arm 210 includes a plurality of links 302 (e.g., linkages). The links 302 (e.g., 302-1 through 302-4) are connected by one or more joints 304 (e.g., 304-1 through 304-5). Each of the joints 304 includes one or more degrees of freedom (DoFs).

In FIG. 6A, the joints 304 include a first joint 304-1 (e.g., a base joint or an A0 joint) that is located at or near a base 306 of the robotic arm 210. In some embodiments, the base joint 304-1 comprises a prismatic joint that allows the robotic arm 210 to translate along the bar 220 (e.g., along the y-axis). The joints 304 also include a second joint 304-2. In some embodiments, the second joint 304-2 rotates with respect to the base joint 304-1. The joints 304 also include a third joint 304-3 that is connected to one end of link 302-2. In some embodiments, the joint 304-3 includes multiple DoFs and facilitates both tilt and rotation of the link 302-2 tilt with respect to the joint 304-3.

FIG. 6A also shows a fourth joint 304-4 that is connected to the other end of the link 302-2. In some embodiments, the joint 304-4 comprises an elbow joint that connects the link 302-2 and the link 302-3. The joints 304 further comprise a pair of joints 304-5 (e.g., a wrist roll joint) and 304-6 (e.g., a wrist pitch joint), which is located on a distal portion of the robotic arm 210.

A proximal end of the robotic arm 210 may be connected to a base 306 and a distal end of the robotic arm 210 may be connected to an advanced device manipulator (ADM) 308 (e.g., a tool driver, an instrument driver, or a robotic end effector, etc.). The ADM 308 may be configured to control the positioning and manipulation of a medical instrument s (e.g., a tool, a scope, etc.).

The robotic arm 210 can also include a cannula sensor 310 for detecting presence or proximity of a cannula to the robotic arm 210. In some embodiments, the robotic arm 210 is placed in a docked state (e.g., docked position) when the cannula sensor 310 detects presence of a cannula (e.g., via one or more processors of the robotic medical system 200). In some embodiments, when the robotic arm 210 is in a docked position, the robotic arm 210 can execute null space motion to maintain a position and/or orientation of the cannula, as discussed in further detail below. Conversely, when no cannula is detected by the cannula sensor 310, the robotic arm 210 is placed in an undocked state (e.g., undocked position).

In some embodiments, and as illustrated in FIG. 6A, the robotic arm 210 includes an input or button 312 (e.g., a donut-shaped button, or other types of controls, etc.) that can be used to place the robotic arm 210 in an admittance mode (e.g., by depressing the button 312). The admittance mode is also referred to as an admittance scheme or admittance control. In the admittance mode, the robotic system 210 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using admittance control, the operator need not overcome all of the inertia in the robotic medical system 200 to move the robotic arm 210. For example, under admittance control, when the operator imparts a force on the arm, the robotic medical system 200 can measure the force and assist the operator in moving the robotic arm 210 by driving one or more motors associated with the robotic arm 210, thereby resulting in desired velocities and/or positions of the robotic arm 210.

In some embodiments, the links 302 may be detachably coupled to the medical tool 212 (e.g., to facilitate case of mounting and dismounting of the medical tool 212 from the robotic arm 210). The joints 304 provide the robotic arm 210 with a plurality of degrees of freedom (DoFs) that facilitate control of the medical tool 212 via the ADM 308. In an embodiment as shown in FIG. 2 including multiple robotic arms, each robotic arm can hold its own respective medical tool and pivot the medical tool about a remote center of motion.

FIG. 6B illustrates a front view of the robotic arm 210. FIG. 6C illustrates a perspective view of the robotic arm 210. In some embodiments, the robotic arm 210 includes a second input or button 314 (e.g., a push button) that is distinct from the button 312 in FIG. 6A, for placing the robotic arm 210 in an impedance mode (e.g., by a single press or continuous press and hold of the button 314). In this example, the button 314 is located between the joint 304-5 and the joint 304-6. The impedance mode is also referred to as impedance scheme or impedance control. In the impedance mode, the robotic medical system 200 measures displacements (e.g., changes in position and velocity) and outputs forces and/or torques to facilitate manual movement of the robotic arm. In some embodiments, the robotic arm 210 can be manually manipulated by a user (e.g., during a set-up procedure) in the impedance mode. In some embodiments, under the impedance mode, the operator's movement of one part of a robotic arm 210 may cause motion in one or more joints and/or links throughout the robotic arm 210.

In some embodiments, for admittance control, a force sensor or load cell can measure the force that the operator is applying to the robotic arm 210 and move the robotic arm 210 in a way that feels light. Admittance control may feel lighter than impedance control because, under admittance control, one can hide the perceived inertia of the robotic arm 210 because motors in the controller can help to accelerate the mass. In contrast, with impedance control, the user is responsible for most if not all mass acceleration, in accordance with some embodiments.

In some circumstances, depending on the position of the robotic arm 210 relative to the operator, it may be inconvenient to reach the button 312 and/or the button 314 to activate a manual manipulating mode (e.g., the admittance mode and/or the impedance mode). Accordingly, under these circumstances, it may be convenient for the operator to trigger the manual manipulation mode other than by buttons.

In some embodiments, the robotic arm 210 includes a single button (e.g., the button 312 or 314) that can be used to place the robotic arm 210 in the admittance mode and/or the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold. In some embodiments, the admittance mode and the impedance mode are common in that they both allow the user to grab the robotic arm 210 and command motion by directly interfacing with it.

In some embodiments, the robotic arm 210 includes an input control for activating an arm follow mode. For example, in some embodiments, the robotic arm 210 can include a designate touch point that is located on a link 302 or a joint 304 of the robotic arm (e.g., an outer shell of the link 302 or a button 316). User interaction (e.g., user touch, contact, etc.) with the designate touch point activates the arm follow mode. In some embodiments, the robotic arm 210 includes multiple touch points. User interaction with any (e.g., one or more) of the touch points activates the arm follow mode.

During a medical procedure, it can be desirable to have the ADM 308 of the robotic arm 210 and/or a remote center of motion (RCM) of the tool 212 coupled thereto kept in a static pose (e.g., position and/or orientation). An RCM may refer to a point in space where a cannula or other access port through which a medical tool 212 is inserted is constrained in motion. In some embodiments, the medical tool 212 includes an end effector that is inserted through an incision or natural orifice of a patient while maintaining the RCM. In some embodiments, the medical tool 212 includes an end effector that is in a retracted state during a setup process of the robotic medical system.

In some circumstances, the robotic medical system 200 can be configured to move one or more links 302 of the robotic arm 210 within a “null space” to avoid collisions with nearby objects (e.g., other robotic arms), while the ADM 308 of the robotic arm 210 and/or the RCM are maintained in their respective poses (e.g., positions and/or orientations). The null space can be viewed as the set of joint states through which a robotic arm 210 can move that does not result in movement of the ADM 308 and/or RCM, thereby maintaining the position and/or the orientation of the medical tool 212 (e.g., within a patient). In some embodiments, a robotic arm 210 can have multiple positions and/or configurations available for each pose of the ADM 308.

For a robotic arm 210 to move an instrument to a desired pose in space, in certain embodiments, the robotic arm 210 may have at least six DoFs-three DoFs for translation (e.g., X, Y, and Z positions) and three DoFs for rotation (e.g., yaw, pitch, and roll). In some embodiments, each joint 304 may provide the robotic arm 210 with a single DoF, and thus, the robotic arm 210 may have at least six joints to achieve freedom of motion to position the ADM 308 at any pose in space. To further maintain the ADM 308 of the robotic arm 210 and/or the remote center or motion in a desired pose, the robotic arm 210 may further have at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 each having more than one degree of freedom thereby achieving the additional DoFs for null space motion. Depending on the embodiments, the robotic arm 210 may have a greater or fewer number of DoFs.

Furthermore, in some embodiments, the bar 220 (e.g., adjustable arm support) can provide several degrees of freedom, including lift, lateral translation, tilt, etc. Thus, depending on the embodiments, a robotic medical system can have many more robotically controlled degrees of freedom beyond just those in the robotic arms 210 to provide for null space movement and collision avoidance. In a respective embodiment of these embodiments, the end effectors of one or more robotic arms (and any tools or instruments coupled thereto) and a remote center along the axis of the tool can advantageously maintain in pose and/or position within a patient.

A robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

In some embodiments, the robotic medical system 200 can be configured to perform collision avoidance to avoid collision(s), e.g., between adjacent robotic arms 210, by taking advantage of the movement of one or more redundant joints in a null space. For example, when a robotic arm 210 collides with or approaches (e.g., within a defined distance of) another robotic arm 210, one or more processors of the robotic medical system 200 can be configured to detect the collision or impending collision (e.g., via kinematics). Accordingly, the robotic medical system 200 can control one or both of the robotic arms 210 to adjust their respective joints within the null space to avoid the collision or impending collision. In an embodiment including at least a pair of robotic arms, a base of one of the robotic arms and its end effector can stay in its pose, while links or joints therebetween move in a null space to avoid collisions with an adjacent robotic arm.

FIGS. 7A and 7B illustrate different views of an exemplary robotic arm 350, in accordance with some embodiments. In accordance with aspects of the subject technology, the surgical robotic arm 350 includes a tool drive 352 and a cannula 362 loaded with a robotic surgical tool. FIGS. 7A and 7B show the robotic arm 350 may include links 351 (e.g., link 351-1, link 351-2, link 351-3, link 351-4, and/or link 351-5) and actuated joint modules (e.g., a joint 353, see also joints J1, J2, J3, J4, J5, J6, J7, and J8) for actuating the plurality of links relative to one another. The joint modules may include various types, such as a pitch joint or a roll joint, which may substantially constrain the movement of the adjacent links around certain axes relative to others. Also shown in the exemplary design of FIGS. 7A and 7B is a tool drive 352 attached to the distal end of the robotic arm 350. The tool drive 352 includes a carriage 354 and a stage 356.

FIG. 7A illustrates that in some embodiments, the tool drive 352 may include a cannula 362 coupled to its end to receive and guide a surgical instrument or end effector 360 (e.g., endoscopes, staplers, scalpel, scissors, clamp, retractor, etc.). The surgical instrument (or “tool”) 360 may include an end effector 364 at the distal end of the tool. The plurality of the joint modules of the robotic arm 350 can be actuated to position and orient the tool drive 352, which actuates the end effector 364 for robotic surgeries. The end effector 364 is at a tool shaft end. In other embodiments, the tool shaft end is a tip of a needle or other object. In some embodiments, the tool drive 352 includes a cannula release lever 358 for releasing the cannula 362 from the tool drive 352.

In some embodiments, the robotic arm 350 includes input devices (e.g., buttons, touchpoints, etc.) FIG. 7B illustrates that in some embodiments, the robotic arm 350 includes a clearance adjustment touchpoint 366, an instrument clutch 368, a port clutch 370, a forearm pivot touchpoint 372, and a forearm multipoint touchpoint 374.

FIGS. 7A and 7B illustrate that the link 351-1 includes a first end that is coupled to the joint J1. In some embodiments, the robotic arm 350 includes a J0 joint that is actuates a second end of the link 351-1. In some embodiments, the joint J0 is a table pivot joint and resides under the surgical table top 402 (see FIGS. 3A and 3B). Joint J0 is nominally held in place during surgery. Joints J1 to J5 form a setup or Cartesian arm and are nominally held in place during surgery, so do not contribute to motion during surgical teleoperation. Joints J6 and J7 (see FIG. 8) form a spherical arm that may actively move during surgery or teleoperation. Joint J8 translates the tool 360, such as the end effector 364, as part of a tool driver. Joint J8 may actively move during surgery. Joints J6, J7, and J8 actively position a tool shaft end (e.g., end effector 364) during surgery while maintaining an entry point into the patient at a fixed or stable location (e.g., remote center of motion) to avoid stress on the body wall of the patient. During setup, any of the joints J0-J8 may move. During surgery, the joints J6, J7, and 18 may move subject to hardware or safety limitations on position, velocity, acceleration, and/or torque. The surgical tool 360 may include none, one, or more (e.g., three) joints, such as a joint for tool rotation plus any number of additional joints (e.g., wrists, rotation about a longitudinal axis, or other type of motion). Any number of degrees of freedom may be provided, such as the three degrees from the joints J6, J7, and J8 and none, one, or more degrees from the surgical tool 360.

FIG. 8 illustrates part of the robotic arm 350 and the surgical tool 360 providing six degrees of freedom (DOF). The six DOF correspond to movement of six active joints during teleoperation. In some embodiments, the active joints include three joints on the surgical tool 360—rotation at joint J9, pitch at wrist joint J10, and yaw at wrist joint J11. The active joints include three joints on the robotic arm 350—spherical rotation joint J6, spherical pitch joint J7, and tool translation joint J8. In some embodiments, other joints providing the six degrees of freedom may be used.

In some embodiments, a use can command movement for fewer than six degrees of freedom. For example, in some embodiments, Five, four, or fewer active joints may be provided. In an example of five DOF, the active joints include three joints on the robotic arm 350—spherical rotation joint J6, spherical pitch joint J7, and tool translation joint J8—and two active joints on the surgical tool—rotation at joint J9 and articulation as another joint. In some embodiments, active joint arrangements may be used, such as providing two or fewer DOF on the robotic arm during teleoperation.

### C. Exemplary Surgical Settings.

FIG. 9 illustrates a perspective view of a robotic medical system 200 that includes four robotic arms 210-1, 210-2, 210-3, and 210-4, in accordance with some embodiments. Each of the robotic arms 210-1, 210-2, 210-3, and 210-4 is coupled to a respective surgical tool 602 (e.g., 602-1 through 602-4, which may correspond to instrument 212) via a respective ADM 308 (e.g., tool driver), such as ADMs 308-1 through 308-4. A surgical tool 602 can be inserted into a patient via a respective port 608 located on the patient. As used herein, a port (e.g., a port location, a port of entry, an entry point, a port region, a port area, or a port position, etc.) refers to a position on a patient's body through which a medical tool/instrument (e.g., held by a robotic arm) can be inserted and constrained in motion. In some embodiments, the port corresponds to an incision point (or an incision region) that is made through the skin of the patient to facilitate a medical operation or procedure. In some embodiments, the port corresponds to a natural orifice, such as a mouth of the patient (e.g., for a bronchoscopy procedure). In some embodiments, the port corresponds to a medical device with an opening, placed at the incision point or the natural orifice to allow access to a surgical space through the opening. The view of the patient has been excluded from FIG. 9 in order to enhance the visibility of the robotic arms 210 and the surgical tools 602.

In FIG. 9, the robotic arm 210-2 is coupled to a camera 606. In this example, the camera 606 is coupled to the robotic arm 210—via a medical instrument 602-2 (e.g., an endoscope) (e.g., at a distal end of the medical instrument 602-2). In some embodiments, the camera 606 is a part of the medical instrument 602-2. In some embodiments, the camera 606 can be a standalone device (e.g., not part of a surgical instrument) that is coupled to a robotic arm (e.g., the camera 606 is distinct and separate from a medical instrument). In some embodiments, the camera 606 defines an axis 604 (e.g., an optical axis), which identifies an orientation of the camera 606. The camera 606 (or a scope) provides an image of a surgical site to facilitate control of surgical tools to perform a robotic medical procedure. For example, a robotically controllable endoscope of the robotic system can include a camera positioned at a distal tip thereof. The user can view an image from the camera of the endoscope in a viewer in order to facilitate control of the endoscope and/or other components of the robotic medical system. As another example, the robotic system may include one or more cameras laparoscopically or endoscopically inserted into a patient. The user can view images from the inserted cameras in order to facilitate control of one or more additional robotically controlled medical instruments, such as one or more additional laparoscopically inserted medical instruments.

In some embodiments, the robotic medical system 200 or the robotic medical system 400 includes a coordinate system (e.g., a robot coordinate system, a coordinate frame, a system frame, etc. that may be a Cartesian or non-Cartesian coordinate system), and respective positions of the patient support platform 202, the table top 402, the robotic arms 210, the robotic arms 350, the adjustable arm supports 220, and/or instruments 212 are represented as coordinates (e.g., x-, y-, and z-coordinates) on the coordinate system. For example, the robotic medical system 200 (e.g., one or more processors 380 of the robotic medical system 200 or the robotic medical system 400) may be configured to identify positions and orientations of the patient support platform 202, the table top 402, the robotic arms 210, the adjustable arm supports 220, and/or instruments 212 based on coordinates in the coordinate system.

### D. External Force Regulation for Teleoperation.

A minimally-invasive robotic surgery with teleoperation usually involves more than one robotic arm and other surgical equipment. During surgery, there is a possibility of collision between robotics arms, or between a robotic arm and an external obstacle (e.g., an external object). Accordingly, there is a need for robotic medical systems to operate in a way that avoids collision, and/or mitigate collision when it occurs.

According to some aspects of the present disclosure, robotic control systems can regulate an external force during teleoperation. In some embodiments, a robotic control system (e.g., robotic medical system 200, robotic medical system, 400, and/or a teleoperation console configured to control one or more robotic arms of a robotic medical system, such as robotic arms 210 or robotic arms 350) uses motor current, dynamic modeling, and constrained inverse kinematics to solve a number of problems, including: estimating a torque using motor current, dynamic modeling of the robotic arm, applying a constrained inverse kinematic solver to enforce a variety of behaviors (e.g., end effector pose targets and/or joint position limits), using torque-motion duality to achieve smooth usability during collision, and using the maximal working space of a robotic arm.

In accordance with some embodiments, during teleoperation, the motion command from a master device is converted to an instrument motion (e.g., the instrument is coupled thereto a robotic arm), a process that is also known as inverse kinematics.

In some embodiments, the motion of the instrument can be viewed by a user/surgeon (e.g., via the physician console 240, as an endoscope live video), and can be mitigated by the user when they observe that something unusual is happening. The motion of the rest of the robotic arm, however, may not be visible to the user. Accordingly, in some embodiments, collision between robotic arms or between a robotic arm and an external obstacle can be mitigated via software algorithms (e.g., that are executed by one or more processors 380).

In some embodiments, during teleoperation, a master device motion is used to control the instrument motion. The master device motion is in task space, which is a six DOF motion (e.g., three DOF for linear motion and three DOF for angular motion). In some embodiments, this task space motion command is converted to arm joint command (joint space) using an inverse kinematic method.

When a collision occurs during teleoperation, one or more joints require more torques than they need to follow a commanded trajectory. In such case, a collision and its direction can be calculated. In some instances, by limiting the joint motion in the direction of collision, such that the joint can only move in the direction to reduce the collision force, the amplitude of collision force to the external obstacles can be limited below a predefined threshold.

In some embodiments, joint trajectory command is achieved by torque from an electrical motor. For a particular motor, the relationship of the torque during the teleoperation can be summarized in the following equation:

$${T}_{motor} = {T}_{gravity} + {T}_{dynamic} + {T}_{friction} + {T}_{rcm} + {T}_{tissue} + {T}_{external} \qquad ( 1 )$$

In Equation (1), Tₘₒₜₒᵣ represents a motor torque, or torque from an electrical motor that is used to actuate a joint or a link of a robotic arm. The motor torque has units of Newton-meter (Nm). Equation (1) shows that a number of torque components contribute to the motor torque, including a gravity torque, a dynamic torque, a friction torque, a remote center of motion (RCM) torque, a tissue torque, and/or an external torque.

In some embodiments, Tₘₒₜₒᵣ can be calculated from motor current using Equation (2):

$${T}_{motor} = η * N * {K}_{t} * {I}_{motor} \qquad ( 2 )$$

In Equation (2), η represents motor efficiency, N represents gear ratio, Kₜ refers to the motor torque constant (with units of Nm/A), and Iₘₒₜₒᵣ is the measured motor current and has units of Ampere (A).

Referring back to Equation (1), the term T_{gravity} represents a gravitational torque (e.g., gravity compensation torque). In some embodiments, T_{gravity} is calculated based on the position of the arm using a dynamic model:

$${T}_{gravity} = G ( θ ) \qquad ( 3 )$$

In Equation (1), the term T_{dynamic} represents the dynamic torque, which is a torque required to balance the inertia force and the Coriolis force. In some embodiments, T_{dynamic} is determined based on the position, velocity, and acceleration of the arm.

$${T}_{dynamic} = M ( θ ) * \ddot{θ} + V ( θ , \dot{θ} ) \qquad ( 4 )$$

In Equation (1), the term T_{friction} represents the friction torque. In some embodiments, T_{friction} is calculated from a coulomb friction model:

$${T}_{friction} = \{ \begin{matrix}sign ( T ) * {T}_{stiction} & when \dot{θ} = 0 \\ - {K}_{f} * \dot{θ} & when \dot{θ} \neq 0\end{matrix} \qquad ( 5 )$$

The term T_{rcm} in Equation (1) represents an RCM torque. In some embodiments, T_{rcm} is the maximum RCM torque during teleoperation. In some embodiments, T_{rcm} is derived from a requirement.

In Equation (1), the term T_{Tissue} represents the maximum torque from tissue force at the tool tip. In some embodiments, T_{Tissue} is the required maximum force from handling tissue during teleoperation, which is given by a requirement, which is usually provided as force (N) at the tool tip.

In Equation (1), the term Tₑₓₜₑᵣₙₐₗ represents the torque from an external object. Tₑₓₜₑᵣₙₐₗ is also referred to as an external torque or a contact torque. In accordance with some embodiments, of the present disclosure, Tₑₓₜₑᵣₙₐₗ is unknown and to be regulated.

In some embodiments, the external torque Tₑₓₜₑᵣₙₐₗ can be determined by rearranging Equation (1). This is represented by Equation (6):

$${T}_{external} = {T}_{motor} - {T}_{gravity} - {T}_{dynamic} - {T}_{friction} - {T}_{rcm} - {T}_{tissue} \qquad ( 6 )$$

Furthermore, in some embodiments, the external torque Tₑₓₜₑᵣₙₐₗ can be represented by:

$${T}_{external} = {T}_{teleop} - {T}_{rcm} - {T}_{tissue} \qquad ( 7 )$$

In Equation (7), the teleoperation torque (Tₜₑₗₑₒₚ) can be determined by:

$${T}_{teleop} = {T}_{motor} - {T}_{gravity} - {T}_{dynamic} - {T}_{friction} \qquad ( 8 )$$

In some embodiments, in order to regulate the forces applied to the environment by the robotic arm, the robotic arm stops the motion to avoid further increasing of the external force. In this case, force and motion has a dual relationship:

$$V = 0 , if dot ( {F}_{external} , V ) > 0 , and {F}_{external} \geq {F}_{stop} \qquad ( 9 )$$

In some embodiments, instead of issuing a command that causes the robotic arm to come to an abrupt halt, a command can be issued to cause a velocity of robotic arm to gradually decrease. Equation (10) illustrates an exemplary relationship in which an updated velocity of the robotic arm is computed as a ratio of a current velocity:

$${V}_{updated} = \frac{( {F}_{stop} - {F}_{external} )}{( {F}_{stop} - {F}_{slowdown} )} \times {V}_{current} , \qquad ( 10 )$$

$$when {F}_{slowdown} \leq {F}_{external} < {F}_{stop}$$

In some embodiments, the robotic control system determines (e.g., automatically, in real time, etc.) am updated velocity of a joint of a robotic arm iteratively (e.g., every one millisecond, every three milliseconds, every 250 milliseconds, etc.).

In joint space approach, the maximum external forces, RCM torque, and tissue forces are converted to joint space requirement using Equation (11):

$$\vec{T} = {J}^{{T}^{′}} \vec{F} \qquad ( 11 )$$

In Equation (11), \vec{T} is a joint torque vector (e.g., a 3×1 vector), J^{T} is the linear portion of the Jacobian matrix (e.g., a 3×3) matrix, and \vec{F} is a force vector (e.g., a 3×1 vector).

In the worst-case scenario, the force vector (\vec{F}) is assumed to be aligned with the task space velocity vector and the torque vector is the joint torque. In this scenario, the external force (Fₑₓₜₑᵣₙₐₗ) is treated as obstacle force, thus always opposite to the velocity direction. In some embodiments, the external force can be calculated by:

$${F}_{external} = - \frac{\vec{V}}{\vert \vec{V} \vert} \times {F}_{ext \_ max} \qquad ( 12 )$$

In some embodiments, the RCM torque (T_{rcm}) can be modeled in a similar way:

$${T}_{rcm} = - \frac{\vec{ω}}{\vert \vec{ω} \vert} \times {T}_{rcm \_ max} \qquad ( 13 )$$

In some embodiments, the tissue force can be modeled in a similar way as Fₑₓₜₑᵣₙₐₗ or T_{rcm}.

In some embodiments, because the tool tip (e.g., instrument tip) is located close to the tool translate joint, it is convenient to shift the tissue force onto the tool translate joint. The benefit is that all instruments (e.g., instruments with four, five, or six DOF) can use the same algorithm to regulate external forces and/or torque(s).

In some embodiments, both the external force and RCM torque can be transferred to the tip forces, such that these three terms (Fₑₓₜₑᵣₙₐₗ, T_{rcm}, and Fₜᵢₛₛᵤₑ) can be lumped together as one tip force limit, which are \vec{F}_{slowdown} and \vec{F}ₛₜₒₚ, which can then be converted to joint torques \vec{T}_{slowdown}, and \vec{T}ₛₜₒₚ (e.g., using Equation (11)). Since \vec{T}_{slowdown}, and \vec{T}ₛₜₒₚ is a lumped limit for all three terms mentions above, combined with Equation (7), the following relationship holds:

$${T}_{{teleop}_{i}} < {T}_{{stop}_{i}} \qquad ( 14 )$$

In Equation (14), the term Tₜₑₗₑₒₚ_{ᵢ} represents a teleoperation torque corresponding to an i^{th} joint of a robotic arm, and the term Tₛₜₒₚ_{ᵢ} represents the maximum torque to stop the motion of an i^{th} joint of a robotic arm.

In some embodiments, because Tₜₑₗₑₒₚ is noisy (e.g., due to motor current noise), a low pass filter is applied to the Tₜₑₗₑₒₚ first. This is represented by Equation (15):

$${T}_{teleop} = lpf ( {T}_{teleop} ) \qquad ( 15 )$$

The term lpf(Tₜₑₗₑₒₚ) represents a low pass-filtered torque. In some embodiments, the low pass filter is a first-order butter worth filter. In some embodiments, the low pass filter has a bandwidth of 2 Hz, 1 Hz, 0.75 Hz, or 0.5 Hz.

In some embodiments, with filtered Tₜₑₗₑₒₚ (e.g., after applying the low pass filter), a force ratio (rᵢ) can be computed as:

$${r}_{i} = \{ \begin{matrix}0 , & when {T}_{{teleop}_{i}} < {T}_{{slowdown}_{i}} \\ \frac{{( {T}_{{teleop}_{i}} - {T}_{{slowdown}_{i}} )}^{2}}{{( {T}_{{stop}_{i}} - {T}_{{slowdown}_{i}} )}^{2}} & when {T}_{{slowdown}_{i}} \leq {T}_{{teleop}_{i}} < {T}_{{stop}_{i}} \\ 1 , & when {T}_{{teleop}_{i}} \geq {T}_{{stop}_{i}}\end{matrix} \qquad ( 16 )$$

The sign of the force ratio is determined on the sign of the joint torque:

$${r}_{i} = sign ( {T}_{teleop} ) \times {r}_{i} \qquad ( 17 )$$

The force ratios are then used as a scalar input for the inverse kinematic solver, which forms a new motion bound:

$$[ Δ {qmin}_{i} , Δ {qmax}_{i} ] = [ Δ {qmin}_{i} , {( 1 - {r}_{i} )}^{′} Δ {qmax}_{i} ] , when {r}_{i}^{′} Δ {qmax}_{i} > 0 \qquad ( 18 a )$$

$$[ Δ {qmin}_{i} , Δ {qmax}_{i} ] = [ {( 1 - {r}_{i} )}^{′} Δ {qmin}_{i} , Δ {qmax}_{i} ] , when {r}_{i}^{′} Δ {qmin}_{i} > 0 \qquad ( 18 b )$$

Constrained Inverse Kinematics solver. In some embodiments, the robotic control system computes a joint motion command by solving a constrained linear equation described in Equation (19):

$$\dot{x} = J * \dot{q} , such that Δ {qmin}_{i} \leq ( \dot{q} ) \leq Δ {qmax}_{i} \qquad ( 19 )$$

In Equation (19), \dot{x}=(ν, ω) is a motion command (e.g., a 6×1 vector) in task space, ν is a linear motion command (e.g., a 3×1 vector) and ω is an angular motion command (e.g., a 3×1 vector). In some embodiments, the robotic control system uses a decoupled (linear and angular) solver to solve for linear motion and angular motion independently, such that the external force regulator has less effect on the instrument orientation control.

In some embodiments, the same approach (e.g., as described above with respect to Equations (1) to (19)) can be used for instruments with six, five, or four DOF.

In some embodiments, an alternative approach to external torque/force regulation in task space includes:
Step 1: Calculate task space force/torque using Equation (7) to set static torque/force limit in task space (T_{slowdown}, Tₛₜₒₚ).
Step 2: Compute teleoperation torque in task space using Equation (19):

$${T}_{task \_ space} = {J}^{′} {T}_{joint} \qquad ( 20 )$$

where T_{task_space} is the task space torque at the RCM (e.g., a 6×1 vector), J is the Jacobian matrix (e.g., a 6×n vector), and Tⱼₒᵢₙₜ is the joint torque vector (e.g., n×1 vector).
Step 3: Apply a low pass filter (e.g., having a frequency of 2 Hz, 1 Hz, 0.75 Hz, 0.5 Hz, etc.) to T_{task_space}.
Step 4: Compute the ratio of motion command r(6×1) using Equations (16) and (17).
Step 5: Compute the maximum allowed motion in task space:

$${\dot{x}}_{i} = {r}_{i}^{′} {\dot{x}}_{i} \qquad ( 21 )$$

where, in Equation (20), xᵢ is the task space motion command for the i^{th} element (e.g., i=1, 2, . . . , 6) and rᵢ is the scalar for the i^{th} element.

### E. System Responses to External Force.

Section 2.D above describes a method of estimating an external force (Fₑₓₜₑᵣₙₐₗ) or an external torque (Tₑₓₜₑᵣₙₐₗ) (e.g., during collision), and using the estimated external force or external torque to determine whether to slow and/or stop the motion of the robotic arm.

In some circumstances, there may be downsides to automatically stopping the robotic arm. For example, it may not be clear to a surgeon why the robotic arm is not moving as intended. Sometimes, there may be a clinical need to complete the commanded robotic arm movement even if it results in a collision.

In accordance with some embodiments of the present disclosure, a robotic control system can, in response to determining that a collision has occurred, complement (e.g., by supplementing or replacing) the system behavior described above to improve system usability and performance.

In some embodiments, when the robotic control system detects a mismatch between a user-commanded position and an actual position of the robotic arm, the robotic control system can provide haptic feedback to the user (via a HID of the physician console 240). In some embodiments, the robotic control system allows the robotic arm to follow the command, but indicates the external collision to the user via haptics in one of several ways. For example, in some embodiments, the robotic control system can increase the damping of the HID during movements in the direction of the collision. In some embodiments, the robotic control system can add a spring-like force to push the HID away from the collision. In some embodiments, the strength of the haptic effect can be a function of the estimated external collision force (e.g., the larger the estimated external collision force, the stronger the haptic effect). This informs the user that there is a collision, but still enables them to drive through the collision if they deem it necessary or advisable.

In some embodiments, when the robotic control system detects a mismatch between a user-commanded position and an actual position of the robotic arm, the robotic control system can provide a notification to the user. For example, the notification can be displayed via a display device (e.g., display device 232 and/or display device 242) and/or via a user interface. In some embodiments, the notification can distinguish various reasons that may prevent motion, such as joint limits versus external collision.

In some embodiments, the robotic control system causes one or more user-selectable interface elements (e.g., buttons, icons, etc.) to be displayed alongside the notification. User selection of the interface elements can indicate that the notification has been acknowledged and/or dismissed. For example, a user can select a first interface element acknowledging that they are aware of the collision and are taking action to address the collision. As another example, a user can select a second interface element indicating that they are aware of the collision and are ignoring it.

In some embodiments, the robotic control system provides a visual cue to indicate a direction of motion of the tooltip (e.g., to indicate that a user should move the tooltip in the direction in order to move away from the collision).

In some embodiments, after detecting that a collision has occurred, the robotic control system provides an indication as to whether the collision is an intra-system collision (e.g., between two robotic arms), or a collision between the robotic medical system and an external object, such as between a robotic arm and a person or between a robotic arm and an equipment. In some embodiments, when the collision is an intra-system collision, the robotic control system can suggest how to reposition itself (e.g., how the robotic arms can be re-positioned) to create more spatial margin between the colliding components.

Accordingly, the disclosed systems and/or methods have several advantages over existing systems. For example, feedback (e.g., haptic feedback or visual notifications) to a user informative and helpful, and can empower users to use the robotic medical system in a clinically useful manner (e.g., rather than having the robotic control system proactively prevent certain motions). The disclosed system also improves over predicate systems, which have low torque saturation and do not allow a surgeon to drive through an external collision, even if it may be clinically beneficial or necessary.

### F. Variations in Force Regulation.

### External Force Estimation at Other Locations

Section 2.D. above describes mapping an external force (Fₑₓₜₑᵣₙₐₗ) or its corresponding torque (Tₑₓₜₑᵣₙₐₗ) during teleoperation at tooltips to determine whether to slow and/or stop the motion of the robotic arms. When the external force is caused by collisions, the estimation at tooltips can make it difficult to determine where the collision occurs. It may be more meaningful and informative to estimate external forces at position(s) outside a patient's body, instead of at tooltips or along the tool shaft below RCM.

In some embodiments, external force can be estimated on a robot arm, which is much more likely to be involved in collisions. Such positions may also include any positions at a tooltip, a tool shaft, a tool stage, and a tool driver, such as the back of the tool stage, back of the tool driver, or other positions along the tool driver. The calculated external force can be compared against established thresholds for biomechanical force limits defined by collaborative robotics standards, such as ISO 15066, Table A.2 (e.g., maximum permissible force at abdomen is 110 Nm), to provide sensible safety coverage at various levels of severity. The primary application of this feature is to regulate force due to external collisions.

### Real Time Determination of Stop Torque and Slowdown Torque Thresholds

The estimation method and implementation described above utilize predetermined constant values for the maximum expected tissue load Tₜᵢₛₛᵤₑ and thresholds, such as T_{slowdown} and Tₛₜₒₚ during normal procedure. Instead of using predetermined constants, maximum expected force/torques can be estimated in real time based on arm pose and/or motion state. A more nuanced determination on when to slowdown or stop the arm can then be made in response to an external force, such as a collision.

In some embodiments, a torque deadband can be added to the maximum expected tissue load Tₜᵢₛₛᵤₑ to determine whether a respective joint force/torque is within or outside an expected range. A deadband is often refers to a band of input values in the domain of a control system where no action occurs (the output is zero). For example, a real-time calculation of the torque deadband may take into consideration of the model error, which can be a sum of the expected model errors derived from:

$${ε}_{model} = {k}_{g} \cdot {T}_{gravity} + {k}_{f} \cdot {T}_{friction} + {k}_{d} \cdot {T}_{dynamic} \qquad ( 22 )$$

where the k_{g}, k_{f}, and, k_{d}∈[0, 1] are accuracy factors for torques T_{gravity}, T_{friction}, and T_{dynamic}, respectively.

Furthermore, the stop torque threshold Tₛₜₒₚ can be calculated and set in real time based on current arm pose and motion. Due to known sources of uncertainty in the force regulation model, the precision with which an unexpected external force can determine depends on arm pose and motion. Unexpected external torques can be estimated more precisely in some arm poses than in others, therefore, setting the threshold Tₛₜₒₚ in real time allows for better safety mitigation coverage, without increasing the likelihood of false positives. The following lists options on setting the threshold:
1) set stop torque threshold Tₛₜₒₚ⁽¹⁾ assuming collision at back of the tool drive stage happening to arm poses in the worst cases. This setting may raise false positives but bring best safety coverage.
2) set stop torque threshold Tₛₜₒₚ⁽²⁾ based on actual arm pose and assume collision at back of the tool drive stage, which may lower false positives but provide less safety coverage.
3) set stop torque threshold based on:

$${T}_{stop}^{( 3 )} = {min}_{T} ( {T}_{stop}^{( 2 )} , {k}_{margin} \cdot ( {T}_{tissue} + {ε}_{model} ) ) \qquad ( 23 )$$

where k_{margin}≥1 is a constant, such as 2.0. Tₛₜₒₚ⁽³⁾ combines Tₛₜₒₚ⁽¹⁾ and Tₛₜₒₚ⁽²⁾ from Option 1) and 2), respectively.

Similarly, slowdown torque threshold T_{slowdown} can be estimated in real time instead of relying on predetermined constants. A feasibility analysis has shown that even under the worst case, the maximum external torque due to normal tissue loads (and model error) always falls below the torque caused by collision force at back of the tool drive stage. This is a prerequisite for the external force regulation to be implementable and useful.

In some embodiments, T_{slowdown} can be derived in real time using the following equation:

$${T}_{slowdown} = {max}_{T} ( {min}_{T} ( {T}_{tissue} + {ε}_{model} , {k}_{margin} \cdot {T}_{stop} ) , ( {T}_{tissue} + {ε}_{model} ) ) \qquad ( 24 )$$

where 0≤k_{margin}≤1 is a constant factor, such as 0.9.

In most cases, T_{slowdown} is set to be greater or equal to (T_{tissue_max}+ε_{model}) to lower the false positives for external force regulation, and less than or equal to Tₛₜₒₚ to smooth out the robot slowdown and/or stop due to force regulation, i.e., (T_{tissue_max}+ε_{model})≤T_{slowdown}≤Tₛₜₒₚ. Like Tₛₜₒₚ, unexpected external torque can be determined more precisely in some arm poses than in other poses, setting T_{slowdown} in real time allows for better safety mitigation without causing more false positives.

### Use of Multiple Filters and Thresholds

False positives can be problematic for force regulation because unnecessary slowdown or stop interrupts teleoperation thus affects the surgeon's ability to complete clinical tasks effectively. As shown in Equation (15) above, a low-pass filter is applied to Tₜₑₗₑₒₚ and indirectly to T_{slowdown} and Tₛₜₒₚ. The filter can filter out sensor noise and reduce false positives. However, it may introduce some latency in system response, which is not ideal particularly for sudden and high impact collisions.

In some embodiments, more than one threshold and/or filters can be applied to improve system response for different types of collisions. For instance, in addition to the low-pass filter in Equation (15), a second low-pass filter with a higher cutoff frequency and a higher stop torque threshold can be adopted. As a result, the system can respond faster to a high impact collision due to the higher cutoff frequency. Furthermore, the second filter is less likely to produce a false positive due to the higher stop torque threshold. Therefore, the first low-pass filter, albeit slower, can detect low impact collisions with fewer false positives, while the second filter is faster to respond and can capture high impact collisions. By running both filters in cadence, improved safety coverage can be achieved without increasing likelihood of false positives. Depending on design goals and priorities, any number of filters and corresponding thresholds can be applied, including an option with no filtering at all for the fastest response time.

### Improved Friction Model

Equation (5) above describes a joint friction model used to estimate motor friction torque T_{friction} due to joint velocity, which is a component of expected torque during normal tele-op. Based on the expected torque value, “unexpected” torque, such as those caused by collision can be determined and estimated. Therefore, any improvements to the friction model help reduce model errors and improved precision and accuracy in estimating external forces.

The described Coulomb friction model refers to a simple linear fit of estimated friction based on motor velocity. In reality, friction is a function of more variables than just velocity, some of which are predictable and repeatable. For example, harmonic drives (a type of motor plus transmission used in robotic arm joints) have position dependent friction. A typical harmonic drive may also have cyclic friction peaks, such as 2×, 4×, and 6×, per motor revolution based on the transmission design and assembly. Other motors may have a position dependent motor cogging effect that can be modeled as friction as well. Some joints may have other joint position dependent friction caused by a seal on the output side of the transmission not perfectly assembled. Furthermore, temperature can affect the friction. Friction model can be adjusted accordingly if robot arm joints sense temperature in real time.

The different effects and variables call for friction models other than a linear fit. For instance, a Fourier series applies well to transmission friction and motor cogging based on motor position. A polynomial, or spline fit, may apply well to friction based on joint position or temperature. We can use the following equation to model the viscous friction:

$${F}_{fricton} = [ - {k}_{coef} \cdot {\dot{x}}_{motor} + {k}_{const} \cdot {ℱ}_{trans} ( {x}_{motor} ) + {𝒮}_{seal} ( {x}_{joint} ) ] \cdot {𝒫}_{temp} ( {t}_{motor} ) \qquad ( 25 )$$

where
k_{coef}—friction coefficient
\dot{x}ₘₒₜₒᵣ—motor velocity
k_{const}—friction constant.
[symbol] ₜᵣₐₙₛ(xₘₒₜₒᵣ)—Fourier series of the transmission friction of motor position xₘₒₜₒᵣ
[symbol] ₛₑₐₗ(xⱼₒᵢₙₜ)—spline fit seal friction of joint position xⱼₒᵢₙₜ.
[symbol] ₜₑₘₚ(tₘₒₜₒᵣ)—polynomial fit temperature adjustment of motor temperature tₘₒₜₒᵣ

Motor velocity \dot{x}ₘₒₜₒᵣ, motor position xₘₒₜₒᵣ, joint position xⱼₒᵢₙₜ, and motor temperature tₘₒₜₒᵣ are real time variables that can be measured from motor/joint status.

Regardless of the friction model of choice, the precision and accuracy of the friction estimate can be further improved by calibrating the pertinent parameters, such as k_{coef}, k_{const}, [symbol] ₜᵣₐₙₛ(⋅), and [symbol] ₜₑₘₚ(⋅), and storing the calibrated parameters on non-volatile memory of the respective device. Furthermore, real time measurement of motor position xₘₒₜₒᵣ, joint position xⱼₒᵢₙₜ, and motor temperature tₘₒₜₒᵣ can also help estimate static friction of the system.

### Application of Force Regulation Algorithm to Non-teleoperation Modes

Section 2.D above introduces the force regulation method that applies to teleoperation. However, the method can be extended to any part of the robotic system susceptible to external collisions in any state. Force regulation can be implemented at any joint, such as joint J0-J5 of the setup arm or joint J6-J8 of the spherical arm. The system can also enable the force regulation at any stage of the procedure, such as during teleoperation, pivot positioning, stow, deploy, procedure setup assist and clearance (e.g., arm/table repositioning).

Collision may happen at locations other than tooltip or RCM, e.g., at various joints, such as at joint J0 or J2. Likewise, HIDs may collide with other parts of the surgeon console, such as the other HID or the arm rest bar, while in position control mode. It may also collide with the operator. Hence, external force regulation can be implemented for HID. Potentially applicable modes for force regulation include HID homing (entire HID), and re-alignment (gimbal only).

### Force/Torque Sensor

In addition to estimating torque at electric motors based on motor current, the joint torque can also be directly measured by a load sensor, such as a force and/or torque sensor embedded within the motor. Motor current sensing is usually noisy, and there can be significant variation in motor torque measurement due to imprecise calibration of tolerance. Load sensor, on the other hand, can provide much more accurate and precise measurement of the joint load than estimations based on motor current and torque values.

In summary, disclosed herein are variations of the force regulation process builds and improves upon the methods described in section D. Estimating external force and regulating system response can be achieved in a more clinically sensible way. Improvements includes higher precision, accuracy, responsiveness, and perceptiveness of the algorithm. Extension of the algorithm can be applied to additional system operation modes with respect to managing external collisions and setting harm thresholds.

### G. Exemplary Processes.

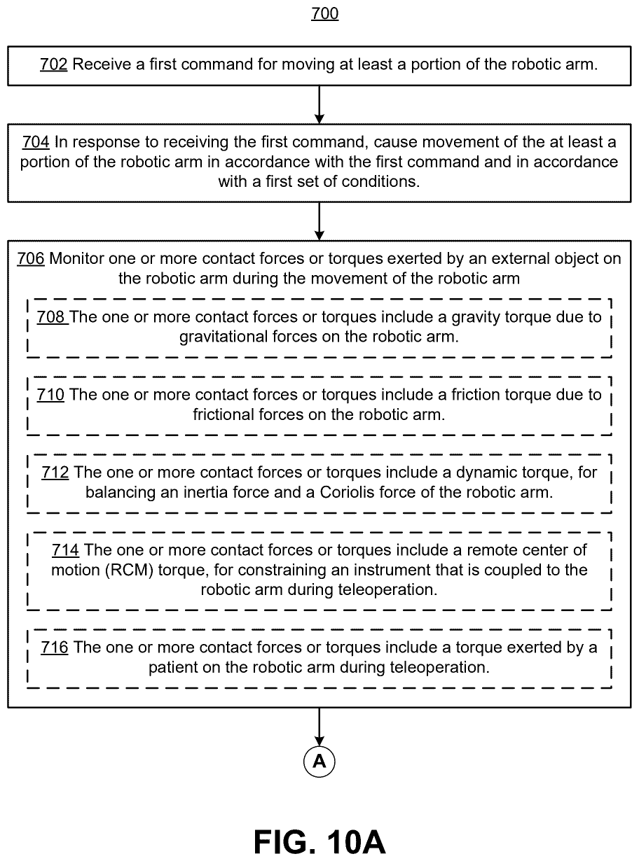

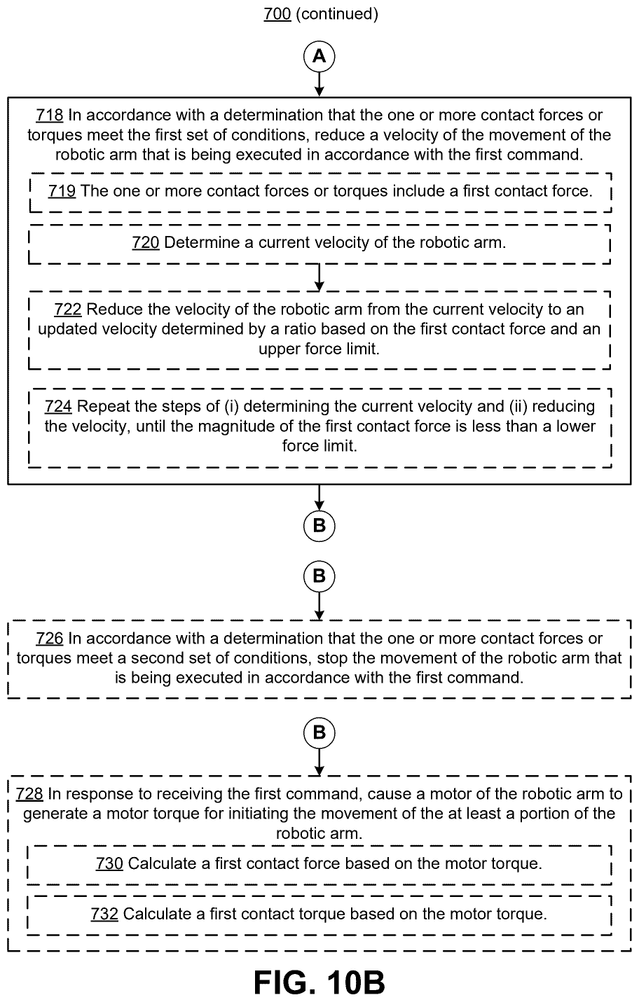

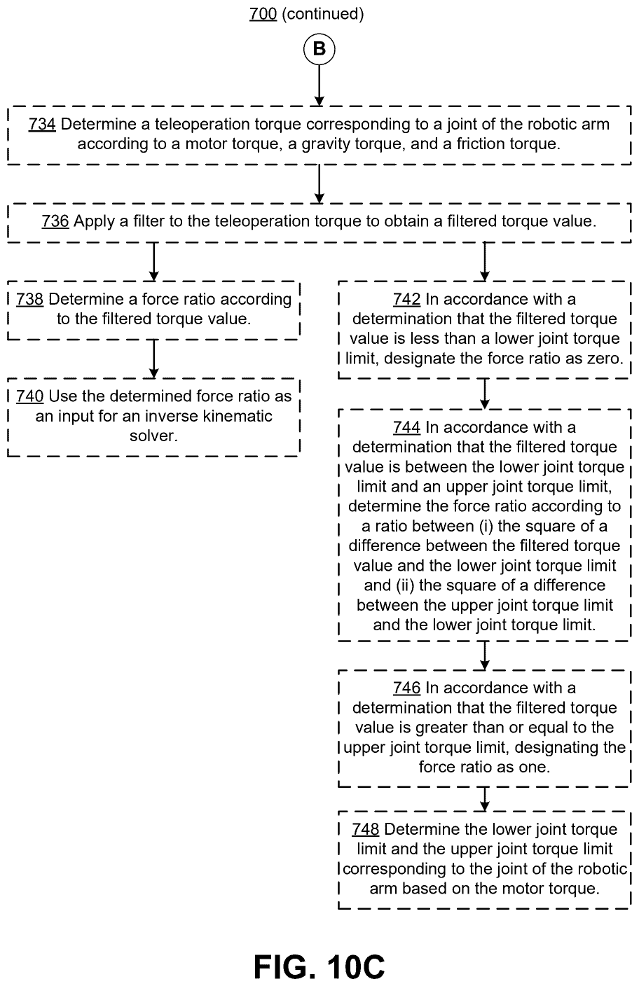

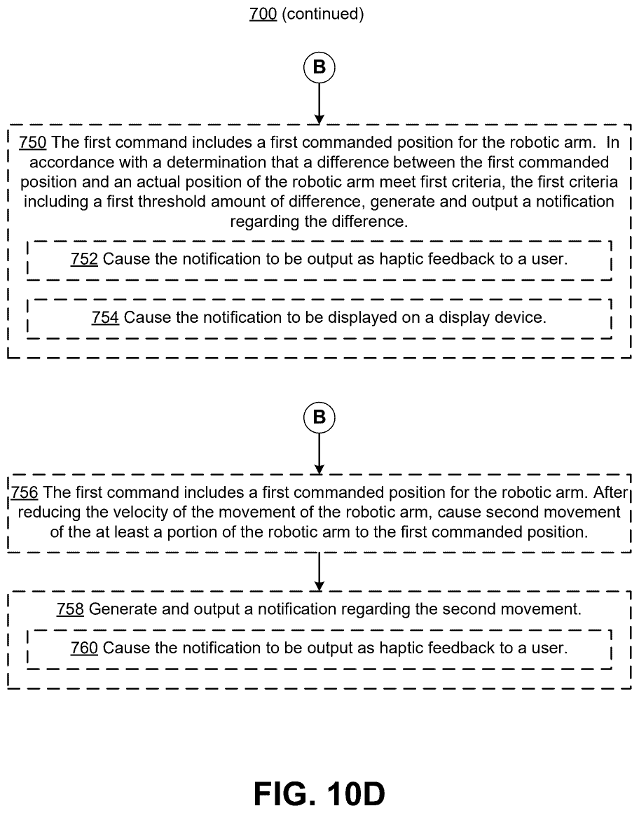

FIGS. 10A to 10D illustrate a flowchart diagram for a method 700 performed by one or more processors (e.g., processors 380) of a robotic control system. In some embodiments, the robotic control system is a robotic medical system (e.g., robotic medical system 200 or robotic medical system 400). In some embodiments, the robotic control system is a teleoperation console (e.g., physician console 240) configured to control one or more robotic arms (e.g., robotic arm 210 or robotic arm 350) of a robotic medical system. The robotic control system includes memory (e.g., memory 382) storing instructions for execution by the one or more processors.

The robotic control system receives (702) a first command for moving at least a portion of (e.g., one or more joints and/or one or more links) the robotic arm.

In some embodiments, the first command includes a user input or a controlled command. For example, in some embodiments, the first command is a motion command provided by (e.g., requested from) an operator (e.g., a surgeon or a surgeon assistant) during teleoperation of the robotic arm. As another example, the first command can include a commanded trajectory, for moving at least a portion of the robotic arm.

In some embodiments, the first command is provided by an operator via a command user interface on a master device or control console (e.g., physician console 240). In some embodiments, the first command is an automatically generated motion command based on preconfigured conditions and desired outcomes.

In some embodiments, the first command is a motion command for moving an instrument attached to the distal end of the robotic arm, and the motion command causes movement of the instrument, and/or movement of one or more joints and/or links of the robotic arm, to achieve the desired motions of the robotic arm and/or the instrument.

In some embodiments, the first command is a motion command for moving the robotic arm in a respective manner. For example, in some embodiments, the first command specifies a displacement of at least a portion of the robotic arm (e.g., move Joint 1 by 5 cm). In some embodiments, the first command specifies movement of the robotic arm to a target pose (e.g., a target position and/or orientation). In some embodiments, the first command specifies a target movement (e.g., a direction and/or a speed) of the at least a portion of the robotic arm.

In some embodiments, the robotic control system, in response to receiving the first command, causes (704) movement of the at least a portion of the robotic arm in accordance with the first command and in accordance with a first set of instructions. For example, in some embodiments, in response to receiving the first command, the robotic control system drives one or more motors (e.g., motor 387-1 or motor 387-2) and/or actuators to cause robotic movement of the robotic arm or to execute a robotic movement of the robotic arm. In some instances, the robotic control system causes movement of at least a portion of the robotic arm (e.g., one or more joints and/or one or more links of the robotic arm) relative to the base or bed of the robotic medical system, relative to a patient, and/or relative to the physical environment, with a respective movement velocity and/or a respective displacement determined in accordance with the first command.

The robotic control system monitors (706) one or more contact forces (e.g., Fₑₓₜₑᵣₙₐₗ) or torques (e.g., Tₑₓₜₑᵣₙₐₗ) (e.g., a contact force or a contact torque) (e.g., an external force or an external torque) exerted by an external object on the robotic arm during the movement of the robotic arm (e.g., while the robotic arm is moving). In some instances, the one or more contact forces or torques is exerted on a joint or a link of the robotic arm.

In some embodiments, the robotic control system measures, estimates, and/or calculates, automatically and in real time, magnitudes and/or directions of the contact forces and torques on the joints and links of the robotic arm, and/or on the instrument(s) attached to the robotic arm).

In some embodiments, the one or more contact forces or torques can include force(s) or torque(s) that are directly detected (e.g., sensed or measured by sensors attached thereto the robotic arms) (e.g., via sensors 388). In some embodiments, the one or more contact forces or torques can include force(s) or torque(s) that are indirectly detected or measured (e.g., calculated or estimated by the robotic control system) (e.g., via processors 380).

In some embodiments, the one or more contact forces or torques include (708) a gravity torque (T_{gravity}) due to gravitational forces on the robotic arm. In some embodiments, the gravity torque is calculated using a dynamic model, based on the position of the robotic arm.

In some embodiments, the one or more contact forces or torques include (710) a friction torque (T_{friction}) due to frictional forces on the robotic arm. This is illustrated in, e.g., Equation (5).

In some embodiments, the one or more contact forces or torques include (712) a dynamic torque (T_{dynamic}), for balancing the inertia force and Coriolis force of the robotic arm. In some embodiments, the dynamic torque is calculated based on the position, velocity, and acceleration of the robotic arm. This is illustrated in Equation (4). In some instances, when the robotic arm is moving slowly, dynamic torque is negligible.

In some embodiments, the one or more contact forces or torques include (714) a remote center of motion (RCM) torque (T_{rcm}), for constraining an instrument (e.g., a tool) that is coupled to the robotic arm during teleoperation (e.g., about a remote center of motion).

In some embodiments, the one or more contact forces or torques include (714) a torque exerted by a patient (e.g., tissue force of a patient) (e.g., T_{Tissue}) on the robotic arm during teleoperation (e.g., a torque from a patient at a tip of an instrument to which the robotic arm is coupled during the teleoperation).

Referring to FIG. 10B, in some embodiments, the robotic control system, in accordance with a determination that the one or more contact forces or torques meet the first set of conditions, reduces (718) a velocity of the movement of the robotic arm that is being executed in accordance with the first command.

In some embodiments, the first set of conditions include: the magnitude of a respective monitored contact force or torque (e.g., tip force, or Tₜₑₗₑₒₚ) is between a lower force or torque limit (e.g., F_{slowdown}, Or T_{slowdown}) and an upper force or torque limit (e.g., Fₛₜₒₚ, or Tₛₜₒₚ). For example, the lower force limit may be any value between 15 N and 40 N. The lower torque limit may be any value from 2 Nm to 4 Nm.

In some embodiments, the first set of conditions include: the direction of a respective monitored contact force or torque (e.g., tip force, or Tₜₑₗₑₒₚ) is in a direction that will result in further increase of the magnitude of the respective monitored contact force or torque beyond a preset threshold.

In some embodiments, the robotic control system, in accordance with a determination that the one or more contact forces or torques meet the first set of conditions, reduces the velocity so that the first set of conditions are no longer met. In some embodiments, the robotic control system, in accordance with a determination that the one or more contact forces or torques meet the first set of conditions, initiates a process to stop the movement of the robotic arm despite of the first command, while maintaining the rigidity of the robotic arm and attached instrument.

In some embodiments, the one or more contact forces or torques include (719) a first contact force (e.g., Fₑₓₜₑᵣₙₐₗ).

Reducing the velocity of the movement of the robotic arm includes determining (720) (e.g., in real-time, automatically and without user intervention) a current velocity (e.g., a real-time velocity) of the robotic arm (e.g., V_{current} in Equation (10)), and reducing (722) the velocity of the robotic arm from the current velocity to an updated velocity (e.g., V_{updated} in Equation (10)) determined by a ratio based on the first contact force (e.g., Fₑₓₜₑᵣₙₐₗ) and an upper force limit (Fₛₜₒₚ)

$$( e . g . , \frac{( {F}_{stop} - {F}_{external} )}{( {F}_{stop} - {F}_{slowdown} )} < 1 ) .$$

For example, the upper force Fₛₜₒₚ may be any value from 45 N to 60 N, in accordance with some embodiments.

In some embodiments, the ratio based on the first contact force and an upper force limit a ratio between (i) a difference between the magnitude of the first contact force and a lower force limit (Fₑₓₜₑᵣₙₐₗ minus F_{slowdown}) and (ii) a difference between the upper force limit and the lower force limit (Fₛₜₒₚ minus F_{slowdown}).

In some embodiments, the robotic control system repeats (e.g., iterates) the steps of (i) determining the current velocity and (ii) reducing the velocity, until the magnitude of the first contact force (e.g., Fₑₓₜₑᵣₙₐₗ) is less than or equal to a lower force limit (e.g., F_{slowdown}) (e.g., when the numerator becomes zero, which means the updated velocity is also zero. See, for example, equation (10)).

In some circumstances, the robotic control system may command all motion to stop if the external force is bigger than safety stop threshold. For example, in some embodiments, the robotic control system, in accordance with a determination that the one or more contact forces or torques meet (726) a second set of conditions, stops the movement of the robotic arm that is being executed in accordance with the first command.

In some embodiments, the second set of conditions include: the magnitude of a respective monitored contact force or torque exceeds (e.g., greater than, greater than or equal to) an upper force limit (e.g., Fₛₜₒₚ) or an upper torque limit (e.g., Tₛₜₒₚ). For example, the upper force Fₛₜₒₚ may be any value from 45 N to 60 N, in accordance with some embodiments. The upper torque limit Tₛₜₒₚ may be any value from 7 Nm to 9 Nm, in accordance with some embodiments.

In some embodiments, in response to receiving the first command, the robotic control system causes (728) a motor of the robotic arm (e.g., a motor coupled to the robotic arm or coupled to a link of the robotic arm) to generate a motor torque (e.g., Tₘₒₜₒᵣ), for initiating the movement of the at least a portion of the robotic arm.

In some embodiments, the motor torque is a torque exerted by the motor on the robotic arm (e.g., on a joint or a link of the robotic arm), to move at least a portion of the robotic arm. In some embodiments, the motor torque needs to overcome/balance the other torques that already exist on the robotic arm, as illustrated in Equation (1).

In some embodiments, for a particular motor, the relationship of the torque during the teleoperation can be summarized in the Equation (1) above.

In some embodiments, the robotic control system calculates (730) (e.g., determines or estimates) (e.g., automatically, repeatedly, without user intervention, etc.) a first contact force (e.g., Fₑₓₜₑᵣₙₐₗ) based on the motor torque. For example, in some embodiments, the robotic control system uses the motor torque (e.g., Tₘₒₜₒᵣ) to determine the external torque (e.g., Tₑₓₜₑᵣₙₐₗ), and obtains (e.g., determines) the external force (e.g., Fₑₓₜₑᵣₙₐₗ) via Equation (11) (e.g., the robotic control system applies an inverse of the Jacobian transpose to obtain the external force).

In some embodiments, the robotic control system calculates (732) (e.g., determines or estimates) (e.g., automatically, repeatedly, without user intervention, etc.) a first contact torque (e.g., Tₑₓₜₑᵣₙₐₗ) based on the motor torque. For example, in some embodiments, the first contact torque can be determined by: Tₑₓₜₑᵣₙₐₗ=Tₜₑₗₑₒₚ−T_{rcm}−Tₜᵢₛₛᵤₑ, where Tₜₑₗₑₒₚ=Tₘₒₜₒᵣ−T_{gravity}−T_{dynamic}−T_{friction}. This is illustrated in, for example, Equations (6), (7), and (8).

With continued reference to FIG. 10C, in some embodiments, the robotic control system determines (734) a teleoperation torque (Tₜₑₗₑₒₚ) corresponding to a joint (e.g., an i^{th} joint) of the robotic arm according to the motor torque, the gravity torque, and the friction torque. This is illustrated in, for example, Equation (8).

In some embodiments, the robotic arm includes a plurality of joints. Each of the joints is electrically coupled to a respective (e.g., distinct) motor. The robotic control system determines a teleoperation torque vector corresponding to the plurality of joints.

In some embodiments, the robotic control system applies (736) a filter to the teleoperation torque to obtain a filtered torque value (e.g., a filtered torque value corresponding to the joint of the robotic arm). In some embodiments, the filter is a low pass filter. In some embodiments, the filter is a first order Butterworth filter. In some embodiments, the filter has a bandwidth of 1 Hz, 0.75 Hz, 0.5 Hz, etc.

In some embodiments, the robotic control system determines (738) (e.g., computes) a force ratio (e.g., rᵢ) according to the filtered torque value, and uses (740) the determined force ratio as an input (e.g., a scalar input) for an inverse kinematic solver, which forms a new motion bound. This is, for example, illustrated in Equations 18(a) and 18(b).

In some embodiments, the robotic control system, in accordance with a determination that the filtered torque value is less than a lower joint torque limit (e.g., \vec{T}_{slowdown}) (the lower joint torque limit is a lower torque limit corresponding to the joint), designates (742) the force ratio as zero. This is, for example, illustrated in Equation (16).

In some embodiments, the robotic control system, in accordance with a determination that the filtered torque value is between the lower joint torque limit and an upper joint torque limit (e.g., \vec{T}ₛₜₒₚ) (the upper joint torque limit is an upper torque limit corresponding to the joint), determines (744) the force ratio according to a ratio between (i) the square of a difference between the filtered torque value and the lower joint torque limit (e.g., (T_{teleop_i}−T_{slowdown_i})²) and (ii) the square of a difference between the upper joint torque limit and the lower joint torque limit. This is, for example, illustrated in Equation (16).

In some embodiments, the robotic control system, in accordance with a determination that the filtered torque value is greater than or equal to the upper joint torque limit, designates (746) the force ratio as one. This is, for example, illustrated in Equation (16).

In some embodiments, the robotic control system determines (748) the lower joint torque limit (e.g., \vec{T}_{slowdown}) and the upper joint torque limit (e.g., \vec{T}ₛₜₒₚ) corresponding to the joint of the robotic arm based on the motor torque.

In some embodiments, due to the fact that tool tip is close to the tool translate joint, it is convenient to shift the tissue force (Tₜᵢₛₛᵤₑ) onto the tool translate joint. Furthermore, both the external force (Fₑₓₜₑᵣₙₐₗ) and RCM torque (T_{rcm}) can be transferred to the tip forces, such that these three terms (Tₜᵢₛₛᵤₑ, Fₑₓₜₑᵣₙₐₗ, and T_{rcm}) can be lumped together as one tip force limit, which are \vec{F}_{slowdown} and \vec{F}ₛₜₒₚ, which can then be converted to joint torques \vec{T}_{slowdown}, and \vec{T}ₛₜₒₚ (e.g., using Equation (11)).

Referring to FIG. 10D, in some embodiments, the first command includes (750) a first commanded position for the robotic arm (e.g., for a joint and/or a link of the robotic arm). In some embodiments, the robotic control system determines an actual position of the robotic arm. In some embodiments, in accordance with a determination that a difference (e.g., a mismatch) between the first commanded position and an actual position of the robotic arm meet first criteria, the first criteria including a first threshold amount of difference, the robotic control system generates and outputs (or causes output of) a notification regarding the difference.

For example, in some embodiments, the difference between the first commanded position and the actual position of the robotic arm includes a difference between the commanded (e.g., target) coordinates and the actual coordinates of a joint of the robotic arm, or an end effector of the robotic arm. or an instrument tip of the robotic arm.

In some embodiments, the notification includes the difference (e.g., coordinates, positional data, etc.) between the first commanded position and the actual position of the robotic arm.)

In some embodiments, the robotic control system causes (752) the notification to be output (e.g., provided) as haptic feedback to a user.

In some embodiments, the haptic feedback can be provided via a human interface device or a haptic interface device (HID). In some embodiments, the HID can be located at a surgeon console. For example, if the robot arm does not follow the user command, the user automatically receives haptic feedback due to the mismatch between the commanded position and the actual position.

In some embodiments, the robotic control system causes (754) the notification to be displayed (e.g., visually) on a display device (e.g., a display device that includes a graphical user interface).

For example, in some embodiments, the display device is a surgeon console that includes a display or a graphical user interface.

In some embodiments, the displayed notification can include and/or distinguish reason(s) as to why motion of the robotic arm is modified or prevented. The reasons can include as joint limits or/versus external collision.

In some embodiments, the notification can include one or more user selectable elements that, when selected by a user, indicates the notification has been acknowledged and/or dismissed (e.g., the user can choose to ignore or address the collision).

In some embodiments, the notification includes a visual cue to indicate the direction the user should move the tooltip in order to move away from the collision.

In some embodiments, based on the system pose (e.g., respective positions and/or orientations of one or more robotic arms), the robotic control system can determine, based on the system pose, whether the collision is likely due to an intra-system collision, or a collision between the system (e.g., between two robotic arms) or due to something external (such as the patient, operating room staff, or other operating room equipment), and provide the determination as part of the notification.

In some embodiments, the first command includes (756) a first commanded position for the robotic arm. After reducing the velocity of the movement of the robotic arm, the robotic control system causes second movement of the at least a portion of the robotic arm to the first commanded position. For example, in some embodiments, the second movement involves movement at the reduced velocity. In some embodiments, the second movement includes continued movement in the first command. That is to say, in some embodiments, the robotic control system allows the robotic arm to follow the first command (e.g., via different conditions or via the same conditions that are specified in the first command).

In some embodiments, the robotic control system generates (758) and outputs (or causes output of) a notification regarding the second movement.

In some embodiments, generating and outputting the notification regarding the second movement includes causing (760) the notification to be output (e.g., provided) as haptic feedback to a user.

In some embodiments, the robotics control system allows the robotic arm to follow the command but indicates the external collision to the user via haptics in several ways. For example, the system can increase the damping of the HID during movements in the direction of the collision.

In some embodiments, the robotics control system generates (or causes the robotic medical system to generate) a spring-like force (or increase a resistance of input controls at the HID) to push the HID away from the collision. In some embodiments, the strength of the haptic effect can be a function of the estimated external collision force. This informs the user that there is a collision, but still enables them to drive through the collision if they deem it necessary.

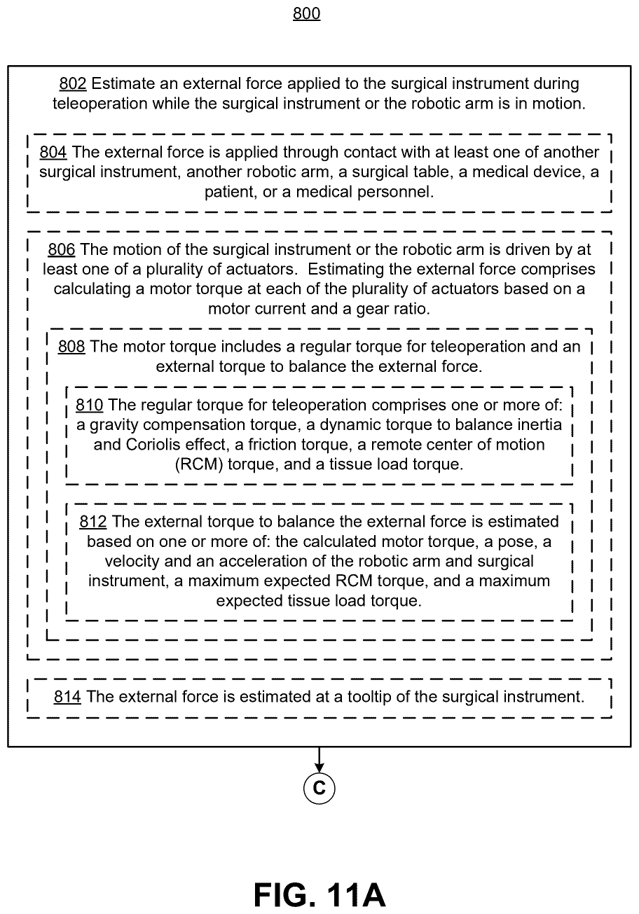

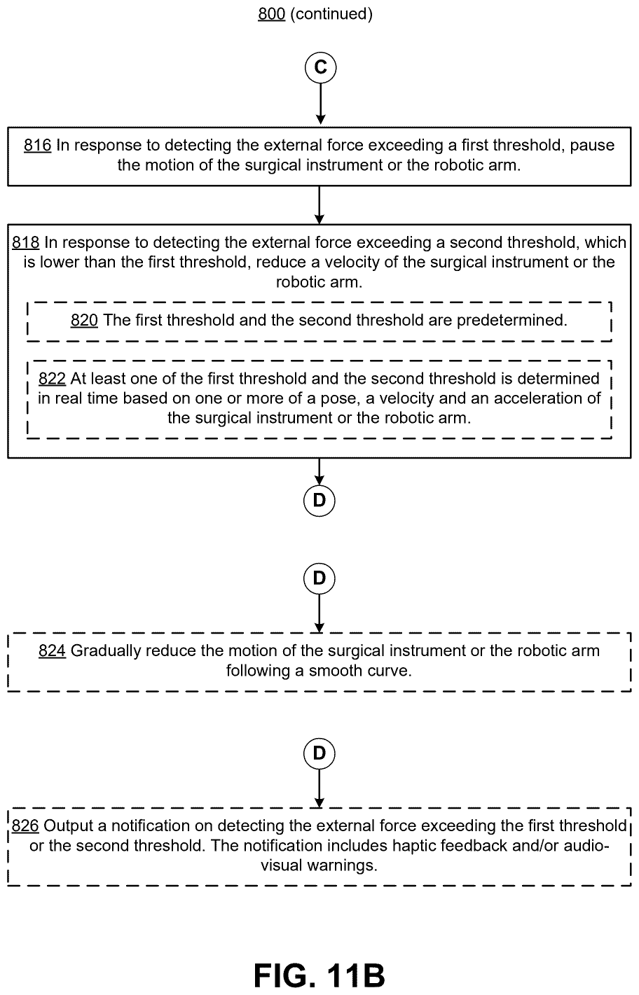

FIGS. 11A and 11B illustrate a flowchart diagram for a method 800 performed by one or more processors (e.g., processors 380) of a surgical robot (e.g., robotic control system or a robotic medical system, such as robotic medical system 200 or robotic medical system 400). In some embodiments, the surgical robot includes a robotic arm. In some embodiments, a surgical instrument is configured to mount on a robotic arm of the surgical robot. The robotic control system includes memory (e.g., memory 382) storing instructions for execution by the one or more processors. Additional details of the method 800 can be found in FIGS. 1 to 10D and the accompanying descriptions, and are not repeated for the sake of brevity.

The surgical robot estimates (802) an external force applied to the surgical instrument during teleoperation while the surgical instrument or the robotic arm is in motion;

In some embodiments, the external force is (804) applied through contact with at least one of another surgical instrument, another robotic arm, a surgical table, a medical device, a patient, or a medical personnel.

In some embodiments, the motion of the surgical instrument or the robotic arm is (806) driven by at least one of a plurality of actuators. Estimating the external force comprises calculating a motor torque at each of the plurality of actuators based on a motor current and a gear ratio.

In some embodiments, the motor torque includes (808) a regular torque for teleoperation and an external torque to balance the external force.

In some embodiments, the regular torque for teleoperation comprises (810) one or more of: a gravity compensation torque, a dynamic torque to balance inertia and Coriolis effect, a friction torque, a remote center of motion (RCM) torque, and a tissue load torque.

In some embodiments, the external torque to balance the external force is (812) estimated based on one or more of the calculated motor torque, a pose, a velocity and an acceleration of the robotic arm and surgical instrument, a maximum expected RCM torque, and a maximum expected tissue load torque.

In some embodiments, the external force is (814) estimated at a tooltip of the surgical instrument.

The surgical robot, in response to detecting that the external force exceeds a first threshold, pauses (816) the motion of the surgical instrument or the robotic arm.

The surgical robot, in response to detecting that the external force exceeds a second threshold, which is lower than the first threshold, reduces (818) a velocity of the surgical instrument or the robotic arm.

In some embodiments, the first threshold and the second threshold are (820) predetermined.

In some embodiments, at least one of the first threshold and the second threshold is (822) determined in real time based on one or more of a pose, a velocity and an acceleration of the surgical instrument or the robotic arm

In some embodiments, the surgical robot (e.g., the one or more processors) gradually reduces (824) the motion of the surgical instrument or the robotic arm following a smooth curve (e.g., a smoothing mathematical relationship (e.g., mathematical function or equation).

In some embodiments, the surgical robot (e.g., the one or more processors) outputs (826) a notification on detecting the external force exceeding the first threshold or the second threshold, wherein the notification includes haptic feedback and/or audio-visual warnings.

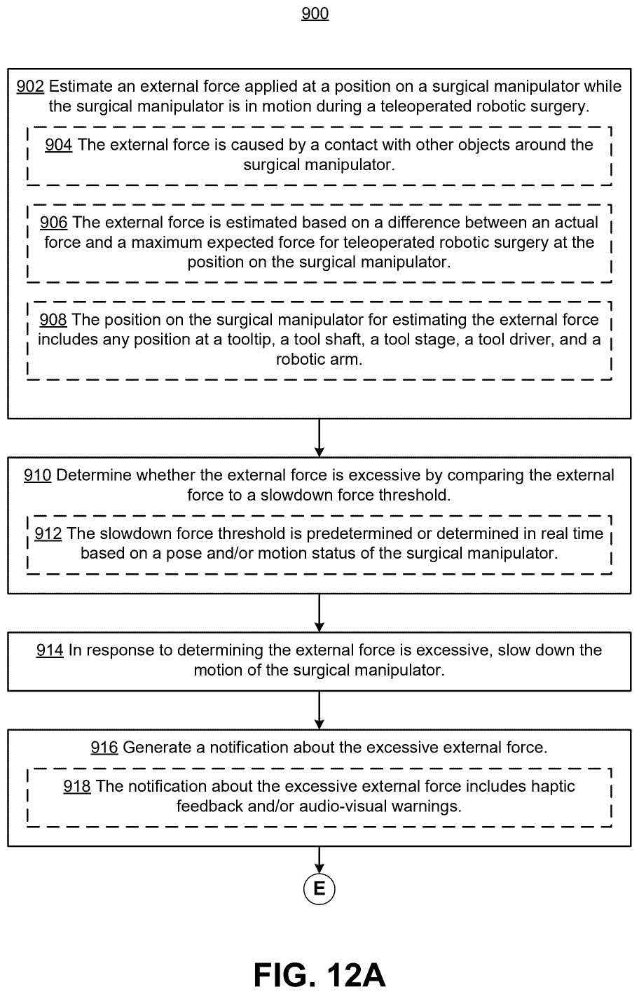

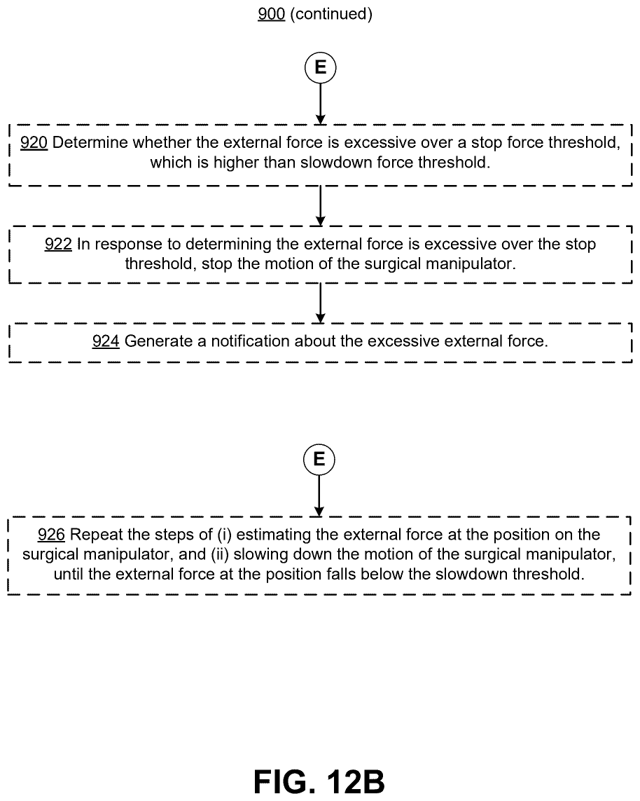

FIGS. 12A and 12B illustrate a flowchart diagram for a method 900. In some embodiments, the method 900 is performed by one or more processors (e.g., processors 380) of a robotic control system or a robotic medical system (e.g., robotic medical system 200 or robotic medical system 400). The robotic control system includes memory (e.g., memory 382) storing instructions for execution by the one or more processors. Additional details of the method 900 can be found in FIGS. 1 to 11B and the accompanying descriptions, and are not repeated for the sake of brevity.

The robotic control system estimates (902) an external force applied at a position on a surgical manipulator while the surgical manipulator is in motion during a teleoperated robotic surgery.

In some embodiments, the external force is (904) caused by a contact with other objects around the surgical manipulator.

In some embodiments, the external force is (906) estimated based on a difference between an actual force and a maximum expected force for teleoperated robotic surgery at the position on the surgical manipulator.

In some embodiments, the position on the surgical manipulator for estimating the external force includes any positions (908) at a tooltip, a tool shaft, a tool stage, a tool driver, and a robotic arm.

The robotic control system determines (910) whether the external force is excessive by comparing the external force to a slowdown force threshold.

In some embodiments, the slowdown threshold is predetermined (912) or determined in real time based on a pose and/or motion status of the surgical manipulator.

The robotic control system, in response to determining the external force is excessive, slows down (914) the motion of the surgical manipulator and generates (916) a notification about the excessive external force.

In some embodiments, the notification about the excessive external force includes (918) haptic feedback and/or audio-visual warnings.

Referring to FIG. 12B, in some embodiments, the method 900 includes determining (920) whether the external force is excessive over a stop force threshold, which is higher than slowdown force threshold. In some embodiments, in response to determining the external force is excessive over the stop threshold, the robotic control system stops (922) the motion of the surgical manipulator and generates (924) a notification about the excessive external force.

In some embodiments, the method 900 includes repeating (926) the steps of (i) estimating the external force at the position on the surgical manipulator, and (ii) slowing down the motion of the surgical manipulator, until the external force at the position falls below the slowdown threshold.

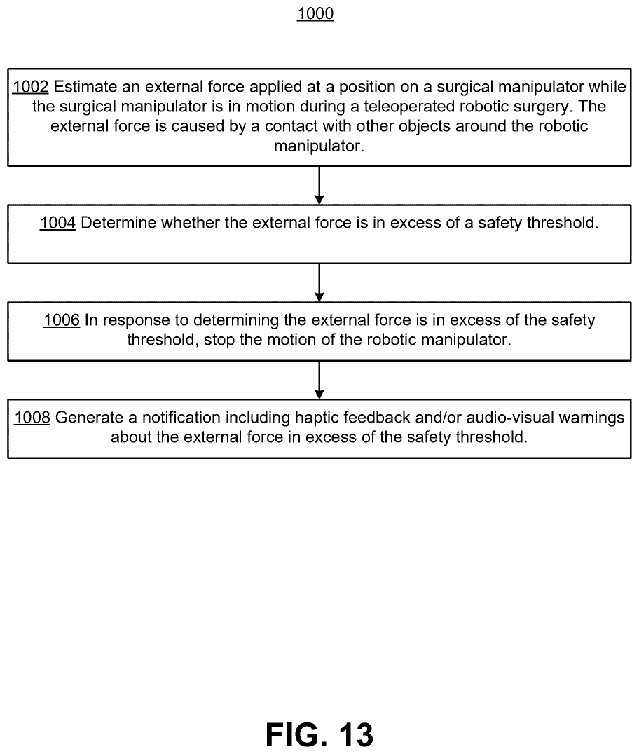

FIG. 13 illustrates a flowchart diagram for a method 1000. In some embodiments, the method 1000 is performed by one or more processors (e.g., processors 380) of a robotic control system or a robotic medical system (e.g., robotic medical system 200 or robotic medical system 400). The robotic control system includes memory (e.g., memory 382) storing instructions for execution by the one or more processors. Additional details of the method 1000 can be found in FIGS. 1 to 12B and the accompanying descriptions, and are not repeated for the sake of brevity.

The robotic control system estimates (1002) an external force applied at a position on a robotic manipulator while the robotic manipulator is in motion during a teleoperated surgery. In some embodiments, the external force is caused by a contact with other objects around the robotic manipulator.

The robotic control system determines (1004) whether the external force is in excess of a safety threshold; and

The robotic control system, in response to determining the external force is in excess of the safety threshold, stops (1006) the motion of the robotic manipulator.

The robotic control system generates (1008) a notification including haptic feedback and/or audio-visual warnings about the external force in excess of the safety threshold

### 3. Implementing Systems and Terminology

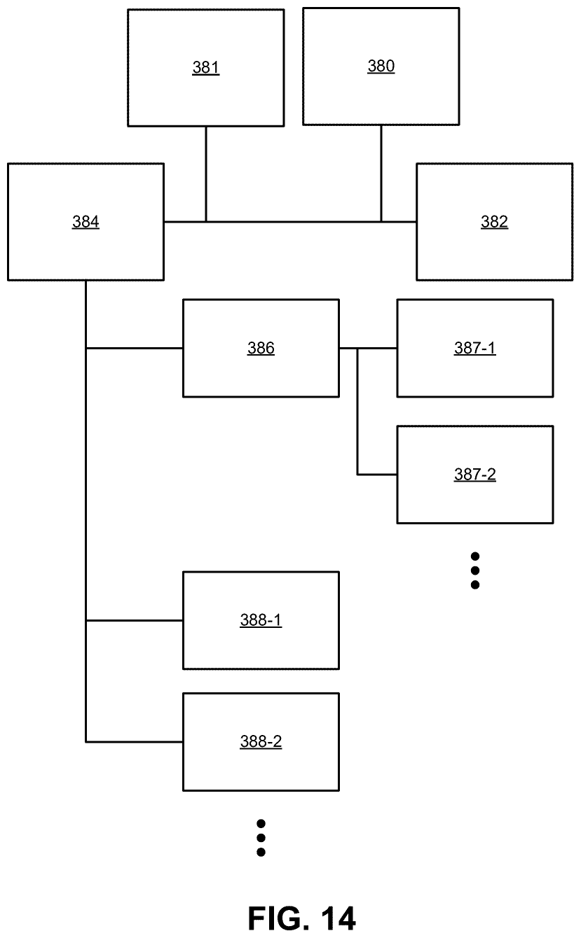

FIG. 14 is a schematic diagram illustrating electronic components of a medical robotic system (e.g., a surgical robotic system) in accordance with some embodiments.

The robotic medical system (e.g., surgical robotic system) includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 1, 2, 3A, 3B, 4, 5, 6A, 6B, 6C, 7A, 7B, 8, 9, 10A, 10B, 10C, 10D, 11A, 11B, 12A, 12B, and 13). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controller 386 are integrated with the input/output controller 384 and the input/output controller 384 provides control signals directly to the one or more actuators 387 (without a separate actuator controller). Although FIG. 14 shows that there is one actuator controller 386 (e.g., one actuator controller for the entire medical robotic system; in some embodiments, additional actuator controllers may be used (e.g., one actuator controller for each actuator, etc.). In some embodiments, the one or more processors 380 are in communication with one or more displays 381 for displaying information as described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the embodiments shown herein but is to be accorded the widest scope consistent with the principles and novel features disclosed herein.

What is claimed is:

1. A surgical robot, comprising:
a surgical instrument configured to mount on a robotic arm; and
a processor configured to:
estimate an external force applied to the surgical instrument during teleoperation while the surgical instrument or the robotic arm is in motion;
in response to detecting the external force exceeding a first threshold, pause the motion of the surgical instrument or the robotic arm; and
in response to detecting the external force exceeding a second threshold, which is lower than the first threshold, reduce a velocity of the surgical instrument or the robotic arm.

2. The surgical robot of claim 1, wherein the external force is applied through contact with at least one of another surgical instrument, another robotic arm, a surgical table, a medical device, a patient, or a medical personnel.

3. The surgical robot of claim 1, wherein the motion of the surgical instrument or the robotic arm is driven by at least one of a plurality of actuators, and
wherein estimating the external force comprises calculating a motor torque at each of the plurality of actuators based on a motor current and a gear ratio.

4. The surgical robot of claim 3, wherein the motor torque includes a regular torque for teleoperation and an external torque to balance the external force.

5. The surgical robot of claim 4, wherein the regular torque for teleoperation comprises one or more of:
a gravity compensation torque,
a dynamic torque to balance inertia and Coriolis effect,
a friction torque,
a remote center of motion (RCM) torque, and
a tissue load torque.

6. The surgical robot of claim 5, wherein the external torque to balance the external force is estimated based on one or more of:
the calculated motor torque,
a pose, a velocity and an acceleration of the robotic arm and surgical instrument,
a maximum expected RCM torque, and
a maximum expected tissue load torque.

7. The surgical robot of claim 1, wherein the external force is estimated at a tooltip of the surgical instrument.

8. The surgical robot of claim 1, wherein the processor is further configured to gradually reduce the motion of the surgical instrument or the robotic arm following a smooth curve.

9. The surgical robot of claim 1, wherein the first threshold and the second threshold are predetermined.

10. The surgical robot of claim 1, wherein at least one of the first threshold and the second threshold is determined in real time based on one or more of a pose, a velocity and an acceleration of the surgical instrument or the robotic arm.

11. The surgical robot of claim 1, wherein the processor is further configured to output a notification on detecting the external force exceeding the first threshold or the second threshold, wherein the notification includes haptic feedback and/or audio-visual warnings.

12. A computer-implemented method, comprising:
estimating an external force applied at a position on a surgical manipulator while the surgical manipulator is in motion during a teleoperated robotic surgery;
determining whether the external force is excessive by comparing the external force to a slowdown force threshold; and
in response to determining the external force is excessive:
slowing down the motion of the surgical manipulator; and
generating a notification about the excessive external force.

13. The computer-implemented method of claim 12, wherein the external force is caused by a contact with other objects around the surgical manipulator.

14. The computer-implemented method of claim 12, wherein the external force is estimated based on a difference between an actual force and a maximum expected force for teleoperated robotic surgery at the position on the surgical manipulator.

15. The computer-implemented method of claim 12, wherein the position on the surgical manipulator for estimating the external force includes any positions at a tooltip, a tool shaft, a tool stage, a tool driver, and a robotic arm.

16. The computer-implemented method of claim 12, wherein the slowdown threshold is predetermined or determined in real time based on a pose and/or motion status of the surgical manipulator.

17. The computer-implemented method of claim 12, further comprising:
determining whether the external force is excessive over a stop force threshold, which is higher than slowdown force threshold; and
in response to determining the external force is excessive over the stop threshold,
stopping the motion of the surgical manipulator; and
generating a notification about the excessive external force.

18. The computer-implemented method of claim 12, further comprising:
repeating the steps of (i) estimating the external force at the position on the surgical manipulator, and (ii) slowing down the motion of the surgical manipulator, until the external force at the position falls below the slowdown threshold.

19. The computer-implemented method of claim 12, wherein the notification about the excessive external force includes haptic feedback and/or audio-visual warnings.

20. A non-transitory computer readable storage medium storing computer-executable instructions, when executed by one or more processors of a robotic control system, cause the one or more processors to perform operations including:
estimating an external force applied at a position on a robotic manipulator while the robotic manipulator is in motion during a teleoperated surgery, the external force caused by a contact with other objects around the robotic manipulator;
determining whether the external force is in excess of a safety threshold; and
in response to determining the external force is in excess of the safety threshold:
stop the motion of the robotic manipulator; and
generating a notification including haptic feedback and/or audio-visual warnings about the external force in excess of the safety threshold.
